(19) United States
(12) Patent Application Publication
(10) Pub. No.: US 2013/0005823 A1
(43) Pub. Date: Jan. 3, 2013

(54) METHODS AND COMPOSITIONS FOR DETERRING ABUSE OF ORALLY ADMINISTERED PHARMACEUTICAL PRODUCTS

(75) Inventors: James F. EMIGH, Palatine, IL (US); Ronald L. LEECH, JR., Plymouth, IN (US); Andrew D. REDDICK, Exton, PA (US); Ron J. SPIVEY, Cooper City, FL (US)

(73) Assignee: ACURA PHARMACEUTICALS, INC., Palatine, IL (US)

(21) Appl. No.: 13/601,239

(22) Filed: Aug. 31, 2012

Related U.S. Application Data
(63) Continuation of application No. 13/450,102, filed on Apr. 18, 2012.
Application No. 13/450,102 is a continuation of application No. 12/001,568, filed on Dec. 12, 2007.
Application No. 12/001,568 is a continuation-in-part of application No. 11/724,502, filed on Mar. 14, 2007, now abandoned.
Application No. 11/724,502 is a continuation-in-part of application No. 11/287,012, filed on Nov. 23, 2005, now abandoned.
Application No. 11/287,012 is a continuation-in-part of application No. 11/136,636, filed on May 24, 2005, now abandoned.
(60) Provisional application No. 60/782,448, filed on Mar. 15, 2006.
(60) Provisional application No. 60/663,973, filed on Mar. 22, 2005.
(60) Provisional application No. 60/643,637, filed on Jan. 13, 2005.
(60) Provisional application No. 60/639,831, filed on Dec. 28, 2004.
(60) Provisional application No. 60/630,991, filed on Nov. 24, 2004.

(51) Int. Cl. A61K 31/137

(52) U.S. Cl. 514/653

(57) ABSTRACT

This invention relates to an abuse deterrent formulation of an oral dosage form of a therapeutically effective amount of any active drug substance that can be subject to abuse combined with a gel forming polymer, a nasal mucosal irritating surfactant and a flushing agent. Such a dosage form is intended to deter abuse of the active drug substance via injection, nasal inhalation or consumption of quantities of the dosage unit exceeding the usual therapeutically effective dose.

### STATEMENT OF RELATED CASES

This application is a continuation of U.S. patent application Ser. No. 13/450,102, filed on Apr. 18, 2012, which in turn is a continuation of U.S. patent application Ser. No. 12/001,568, filed on Dec. 12, 2007, which in turn is a continuation-in-part of U.S. patent application Ser. No. 11/724,502, filed on Mar. 14, 2007, which claims the benefit of U.S. Provisional Application No. 60/782,448 filed on Mar. 15, 2006, and is a continuation-in-part of U.S. patent application Ser. No. 11/287,012 filed on Nov. 23, 2005, which in turn is a continuation-in-part of U.S. patent application Ser. No. 11/136,636, filed on May 24, 2005; and which claims priority to U.S. Provisional Application Nos. 60/693,898 filed on Jun. 24, 2005; 60/663,973, filed on Mar. 22, 2005; 60/643,637, filed on Jan. 13, 2005; 60/639,831, filed on Dec. 28, 2004; and 60/630,991, filed on Nov. 24, 2004. The content of each of the preceding documents is hereby incorporated by reference in its entirety.

### FIELD OF THE INVENTION

This invention pertains to compositions and methods of formulating dosage forms (e.g., orally administered pharmaceutical products) containing one or more active pharmaceutical ingredients susceptible to abuse, including, but not limited to, opioid analgesics such that the resulting dosage form is abuse deterrent.

### BACKGROUND OF THE INVENTION

The class of drugs exhibiting opium or morphine-like properties are referred to as opioids, or opioid agonists. Certain opioids act as agonists, interacting with stereo specific and saturable binding sites in the brain and other body tissues and organs. Endogenous opioid-like peptides are present in areas of the central nervous system that are presumed to be related to the perception of pain; to movement, mood and behavior; and to the regulation of neuroendocrinological functions. Three classical opioid receptor types, mu (μ), delta (δ), and kappa (κ), have been studied extensively. Each of these receptors has a unique anatomical distribution in the brain, spinal cord, and the periphery. Most of the clinically used opioids are relatively selective for μ receptors, reflecting their similarity to morphine. However, opioid containing drugs that are relatively selective for a particular receptor subtype at standard therapeutic doses will often interact with multiple receptor subtypes when given at sufficiently high doses, leading to possible changes in their pharmacological effect. This is especially true as opioid doses are escalated to overcome tolerance.

The potential for the development of tolerance, physical and/or psychological dependence (i.e., addiction) with repeated opioid use is a characteristic feature of most drugs containing opioid analgesics. The possibility of developing addiction is one of the major concerns in the use of opioids for the management of pain. Another major concern associated with the use of opioids is the diversion of these drugs from a patient in legitimate pain to other individuals (non-patients) for recreational purposes.

Drug abusers and/or addicts typically may take a solid dosage form intended for oral administration containing one or more opioid analgesics and crush, shear, grind, chew, dissolve and/or heat, extract or otherwise tamper with or damage the dosage unit so that a significant portion or even the entire amount of the active drug becomes available for administration by 1) injection, 2) inhalation, and/or 3) oral consumption in amounts exceeding the typical therapeutic dose for such drugs.

There are three basic patterns of behavior leading to opioid abuse. The first involves individuals whose opioid drug use begins in the context of legitimate medical treatment and who obtain their initial drug supplies through prescriptions from appropriately licensed health care providers. Through an insidious process these individuals may ultimately begin seeking prescription drug supplies far exceeding their legitimate medical needs from multiple health care providers and/or pharmacies and/or from illicit sources diverted from otherwise legal drug distribution channels. The second pattern of abuse begins with experimental or “recreational” drug users seeking a “high” with no legitimate medical indication for drugs subject to abuse. A third pattern of abuse involves users who begin in one or another of the preceding ways and ultimately switch to orally administered opioids such as methadone, obtained from organized and legitimate addiction treatment programs.

There are various routes of administration an abuser may commonly employ to abuse an opioid containing drug formulation. The most common methods include 1) parenteral (e.g. intravenous injection), 2) intranasal (e.g., snorting), and 3) repeated oral ingestion of excessive quantities, for example, of orally administered tablets or capsules. One mode of abuse of oral solid drugs involves the extraction of the opioid component from the dosage form by first mixing the dosage form with a suitable solvent (e.g., water), and then subsequently extracting the opioid component from the mixture for use in a solution suitable for intravenous injection of the opioid to achieve a “high.”

Attempts have been made to diminish the abuse potential of orally administered opioid drugs. These attempts generally centered on the inclusion in the oral dosage form of an opioid antagonist which is not orally active but which will substantially block the analgesic effects of the opioid if one attempts to dissolve the opioid and administer it parenterally.

For example, commercially available Talwin®Nx tablets, indicated for the relief of moderate to severe pain, contain a combination of pentazocine and naloxone. Pentazocine is a partial agonist of μ receptors and also has affinity for κ receptors. Naloxone is an antagonist of μ receptors. The amount of naloxone present in this combination has no action when taken orally, and will not interfere with the pharmacologic action of pentazocine. However, this amount of naloxone given by injection has profound antagonistic action to opioid analgesics. Thus, the inclusion of naloxone is intended to curb the abuse of oral pentazocine which occurs when the oral dosage form is solubilized and injected. Therefore, this combination dosage form has lower potential for parenteral misuse than single entity oral pentazocine formulations. Several patents describe abuse deterrent formulations, including the following.

U.S. Pat. No. 6,559,159 (Carroll et al.) describes the use of kappa receptor antagonists for the treatment of opioid related addictions. One such commercially available product is naltrexone tablets indicated for blocking the effects of exogenously administered opioids.

U.S. Pat. No. 6,375,957 (Kaiko et al.) describes the combination of an opioid agonist, a non-steroidal anti-inflammatory drug, and an orally active opioid antagonist. The purpose of the orally active opioid antagonist is the same as discussed above.

U.S. Pat. No. 4,457,933 (Gordon et al.) describes a method for decreasing both the oral and parenteral abuse potential of analgesics such as oxycodone, propoxyphene and pentazocine by combining an analgesic dose of the analgesic agents with naloxone in specific, relatively narrow ranges.

U.S. Pat. No. 6,228,863 B1 (Palermo et al.) describes a method for reducing the abuse potential of an oral dosage form of an opioid analgesic, whereby an orally active opioid agonist is combined with an opioid antagonist into an oral dosage form requiring at least a two-step extraction process to be separated from the opioid agonist, the amount of opioid antagonist included being sufficient to counteract opioid effects if extracted together with the opioid agonist and administered parenterally.

U.S. Pat. No. 6,593,367 (Dewey et al.), describes a method whereby the addiction-related behavior of a mammal suffering from addiction could be changed by a combination of drugs. The method includes administering to the mammal of an effective amount of gamma vinyl GABA (GVG) or a pharmaceutically acceptable salt, or an enantiomer or a racemic mixture, where the effective amount is sufficient to diminish, inhibit or eliminate behavior associated with craving or use of the combination of abused drugs. U.S. Pat. Nos. 4,175,119 and 4,459,278 (Porter et al.) describe compositions and methods useful for the prevention of accidental and/or intentional oral overdoses of a drug.

In summary, various attempts have been made and are described in the prior art to develop abuse deterrent dosage forms. Despite all attempts, the misuse and abuse of pharmaceutical products continues to increase. Clearly there is a growing need for novel and effective methods and compositions to deter abuse of pharmaceutical products (e.g., orally administered pharmaceutical products) including but not limited to immediate release, sustained or extended release and delayed release formulations for drugs subject to abuse. In particular, such methods and compositions would be useful for opioid analgesics, for patients seeking drug therapy, which deter abuse and minimizes or reduces the potential for physical or psychological dependency.

### SUMMARY OF THE INVENTION

The present invention includes a pharmaceutical composition (e.g., an oral solid pharmaceutical product) of any active drug substance susceptible to abuse, a gel forming polymer, a surfactant in sufficient amounts to cause nasal or mucosal irritation, and an agent in sufficient amounts to cause flushing, or other unpleasant peripheral vasodilatory effects, if the amount of the active drug subject to abuse is ingested in amounts exceeding the usual recommended therapeutic dose.

In one embodiment, the therapeutic pharmaceutical composition can be formed into a unit dose including an opioid analgesic, a gel forming polymer, a nasal tissue irritating amount of a surfactant, and a flushing agent in sufficient amount to cause flushing if greater than a prescribed amount of the analgesic included in the therapeutic composition is ingested. In one embodiment, the polymer includes one or more of polyethylene oxide (e.g., having average molecular weight ranging form about 300,000 to about 5,000,000), polyvinyl alcohol (e.g., having a molecular weight of about 20,000 to 200,000), hydroxypropyl methyl cellulose (e.g., having a molecular weight of about 10,000 to 1,500,000), and a carbomer (e.g., having a molecular weight ranging of about 700,000 to 4,000,000,000), the nasal irritant includes about 1 to 5 percent by weight sodium lauryl sulfate, and the flushing agent includes about 0.01 to 0.5 gm of niacin.

The present invention also provides methods of making a pharmaceutical composition suitable for deterring drug abuse including one or more steps of providing an analgesic, a gel forming polymer having a suitable viscosity, a nasal tissue irritant and a flushing agent, controlling the molecular weight or viscosity of the gel forming polymer to form a gel, controlling the amount of nasal tissue irritant such that nasal tissue irritation occurs if inhaled, controlling the amount of flushing agent such that flushing ensues only if more than a prescribed amount of the analgesic is consumed, and combining the analgesic, gel forming polymer, nasal tissue irritant and flushing agent to form a therapeutic composition.

The present invention also includes a therapeutic pharmaceutical composition including an analgesic, a gel forming polymer, a surfactant present in sufficient amount to cause nasal irritation, and an agent in sufficient amount to cause emesis if greater than a prescribed amount of the analgesic included in the therapeutic composition is ingested. The present invention also includes a therapeutic pharmaceutical composition including an opioid analgesic, a gel forming polymer, a surfactant present in sufficient amount to cause nasal irritation, and an emetic in sufficient amount to cause emesis if greater than a prescribed amount of the analgesic included in the therapeutic composition is ingested.

In one embodiment, the therapeutic pharmaceutical composition can be formed into a unit dose including an opioid analgesic, a gel forming polymer, a nasal tissue irritating amount of a surfactant, and an emetic in sufficient amount to cause emesis if greater than a prescribed amount of the analgesic included in the therapeutic composition is ingested. In one embodiment, the polymer includes one or more of polyethylene oxide (e.g., having average molecular weight ranging form about 300,000 to about 5,000,000), polyvinyl alcohol (e.g., having a molecular weight of about 20,000 to 200,000), hydroxypropyl methyl cellulose (e.g., having a molecular weight of about 10,000 to 1,500,000), and a carbomer (e.g., having a molecular weight ranging of about 700,000 to 4,000,000,000), the nasal irritant includes about 1 to 5 percent by weight sodium lauryl sulfate, and the emetic includes less than about 0.6 to 2.0 gm of zinc sulfate.

The present invention also provides methods of making a pharmaceutical composition suitable for deterring drug abuse including one or more steps of providing an analgesic, a gel forming polymer having a suitable viscosity, a nasal tissue irritant and emetic, controlling the molecular weight or viscosity of the gel forming polymer to form a gel of a desired viscosity upon combination with a solvent, controlling the amount of nasal tissue irritant such that nasal tissue irritation occurs if inhaled, controlling the amount of emetic such that emesis ensues only if more than a prescribed amount of the analgesic is consumed, and combining the analgesic, gel forming polymer, nasal tissue irritant and emetic to form a therapeutic composition.

The present invention includes a therapeutic pharmaceutical composition including an analgesic, a gel forming polymer, a surfactant present in sufficient amount to cause mucosal tissue irritation, and a flushing agent in sufficient amount to cause flushing if greater than a prescribed amount of the analgesic included in the therapeutic composition is ingested.

Compositions and methods of the present invention can deter abuse of the analgesic by forming a viscous gel upon contact with a solvent such that the gel and analgesic cannot be easily drawn into a syringe and/or by inducing nasal irritation if the composition is inhaled, and/or by inducing emesis and/or flushing and/or nasal and/or sinus blockage if more than a prescribed dosage amount of the analgesic is consumed or if the dosage form is administered in a manner inconsistent with a manner suggested by a healthcare provider.

In one embodiment, the present invention includes one or more abuse deterrents selected from the group of overall deterrent classes including: gel forming agents, tissue (e.g., mucous membrane) irritants, emetics, stool softeners, tissue staining agents, malodorous/repugnant agents, flushing agents and pain or discomfort causing agents, for example as set forth below in sections B through H.

In some embodiments, the agents included in the present invention are generally considered safe when administered at levels that are less than the threshold amount for each particular agent. The threshold amounts for each particular agent are described in more detail below. In certain embodiments, when administered in an amount which is less than the threshold amount, an agent included in the present invention can have no abuse deterrent effect or a beneficial effect on a subject.

In some embodiments, the present invention provides a composition and a method for making a therapeutic pharmaceutical composite suitable for deterring drug abuse including at least two immediate release sequestered subunits including: a first immediate release sequestered subunit including one or more abuse deterrent agent, and a second immediate release sequestered subunit including a drug susceptible to abuse. In one embodiment, the present invention has at least two immediate release sequestered subunits including: a first immediate release sequestered subunit including a mixture of: a gel forming polymer, a nasal tissue irritant, and a disintegrant; and a second immediate release sequestered subunit including an opioid analgesic.

In some embodiments, the present invention includes a sequestered pharmaceutical composition subunit including: a gel forming polymer, a nasal tissue irritant, and a disintegrant. In one embodiment, the present invention includes a sequestered pharmaceutical composition subunit including: polyethlene oxide in an amount of about 3 to about 40 percent by weight; sodium lauryl sulfate in an amount of about 1 to about 10 percent by weight; and crosprovidone in an amount of about 2 to about 25 percent by weight.

### BRIEF DESCRIPTION OF THE DRAWINGS

The present invention will be better understood by examining the following figures which illustrate certain properties of the present invention wherein:

FIG. 1 shows a percentage amount of certain opioid drugs available in solution for injection after certain embodiments of standard dosage forms are crushed and exposed to a solvent;

FIG. 2 shows a percentage amount of certain opioid drugs available in solution for injection after dosage forms of the present invention are crushed and exposed to a solvent;

FIG. 3 shows an amount of drug recoverable from a solvent contacted with five embodiments of the present invention compared to a standard formulation;

FIG. 4 shows a dissolution profile of six embodiments of the present invention;

FIG. 5a shows various dosage forms having one or more abuse deterrent properties of the present invention;

FIG. 5b shows a particular dosage form having one or more abuse deterrent properties of the present invention;

FIG. 5c shows a particular dosage form having one or more abuse deterrent properties of the present invention and a disintegrant;

FIG. 6 shows a process flow chart for one embodiment of the manufacture of a dosage form of the present invention;

FIG. 7 shows a dissolution profile of three extended release formulations of the present invention;

FIG. 8 shows a dissolution profile of several embodiments of tablets according to the present invention for prior art compositions, and certain embodiments of compositions according to the present invention containing oxycodone;

FIG. 9 shows the effect of micro crystalline cellulose (Avicel) on dissolution for certain embodiments of compositions according to the present invention compared to known compositions; and

FIG. 10 shows the percent subjects having symptoms induced by a flushing/pain/headache inducing agent of the invention.

With reference to the Figures, features that are the same across the Figures are denoted with the same reference numbers.

### DETAILED DESCRIPTION OF THE INVENTION

The present invention includes an abuse deterrent formulation for reducing the potential for one or more of a) parenteral abuse, b) inhalation (e.g., by the nasal or oral respiratory route), and/or c) oral abuse of a drug, typically an opioid analgesic type drug, for satisfaction of a physical or psychological dependence.

In one embodiment, the present invention includes one or more abuse deterrents selected from the group of overall deterrent classes including: gel forming agents, tissue (e.g., mucous membrane) irritants, emetics, stool softeners, tissue staining agents, malodorous/repugnant agents, flushing agents and pain or discomfort causing agents, for example as set forth below in sections B through H. In one embodiment, the present invention includes two or more deterrents, each selected from a different class of deterrent (e.g., an emetic and gel forming agent). In another embodiment, the present invention includes at least three or more, potentially four or more deterrents, each selected from a different class of deterrent (e.g., a flushing agent, a gel forming agent, and a tissue staining agent).

In another embodiment, the present invention can include one or more deterrents selected from the group of deterrent classes set forth above, and wherein multiple deterrents can be selected from within the same class (e.g., one or more different gel forming agents combined with one or more different flushing agents and/or combined with one or more irritants). The selection of the number and/or type of each overall class of deterrent, as well as the selection of the number and/or type of particular deterrent within each class to be used in a pharmaceutical containing dosage form of the present invention, is selected to deter one or more particular forms of abuse and is believed to be within the skill of the artisan upon reading this disclosure.

In one embodiment, the present invention deters parenteral abuse by providing a pharmaceutical composition which includes a therapeutically active pharmaceutical, and in particular one or more therapeutically active pharmaceuticals which are susceptible to abuse (e.g., analgesics) with one or more gel forming agents such that upon contact with a solvent (e.g., water), the agents swell by absorbing the solvent thereby 1) entrapping the drug in a gel matrix and/or 2) reducing or preventing a significant amount of the opioid analgesic from being drawn into a syringe. In one embodiment, the present invention deters inhalation abuse by providing a pharmaceutical composition which includes a therapeutically active pharmaceutical (e.g., an analgesic), and one or more mucous membrane, mucosa or mucosal tissue irritants (collectively referred to as mucous membrane irritants). In one embodiment, the mucosal tissue is nasal passageway tissue.

Upon contact with a mucous membrane, the irritants induce temporary discomfort, pain and/or irritation of the membranes and/or tissues to thereby deter abuse. For example, if inhaled by snorting, the mucous membrane in the nasal passageway will be irritated and result in significant discomfort and/or pain to the individual. Additionally, nasal and/or sinus blockage may occur if a gel forming agent is present. In one embodiment, the present invention provides a pharmaceutical composition which includes an analgesic with one or more emetics, such that after oral consumption of more than a typically prescribed amount of the dosage form, emesis is induced.

In one embodiment, two or more of the abuse deterrents from a single class of deterrents and/or from multiple classes of deterrents can be combined into one composition according to the present invention. In another embodiment, three or more of the abuse deterrents from a single class of deterrents and/or from multiple classes of deterrents can be combined into one composition according to the present invention.

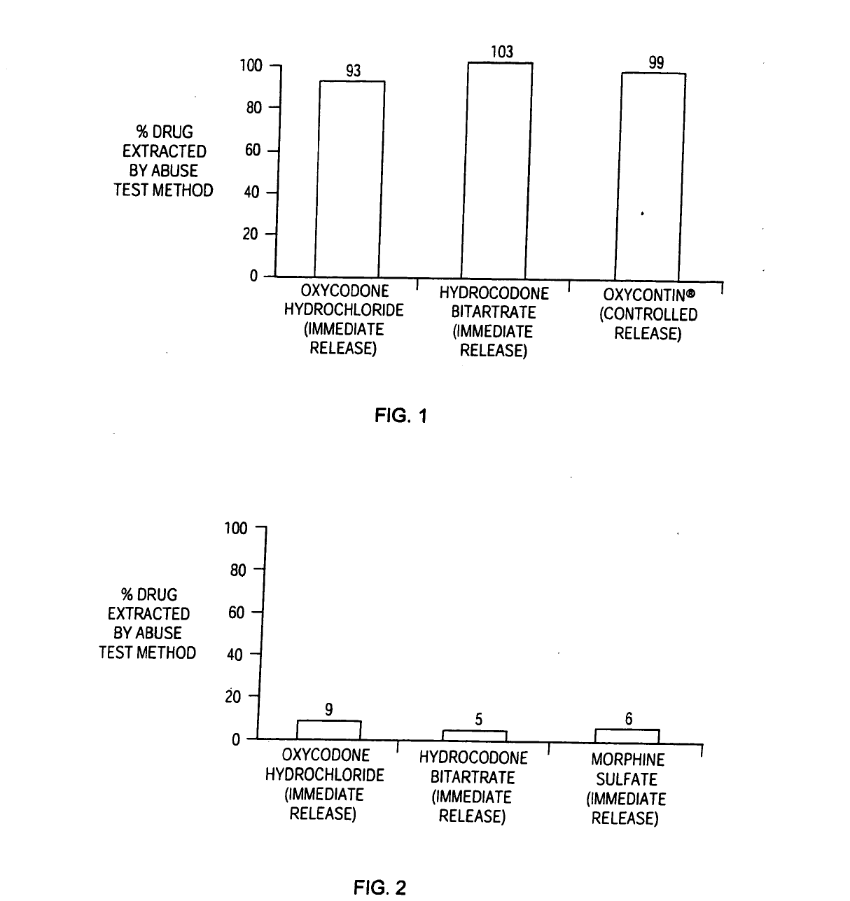

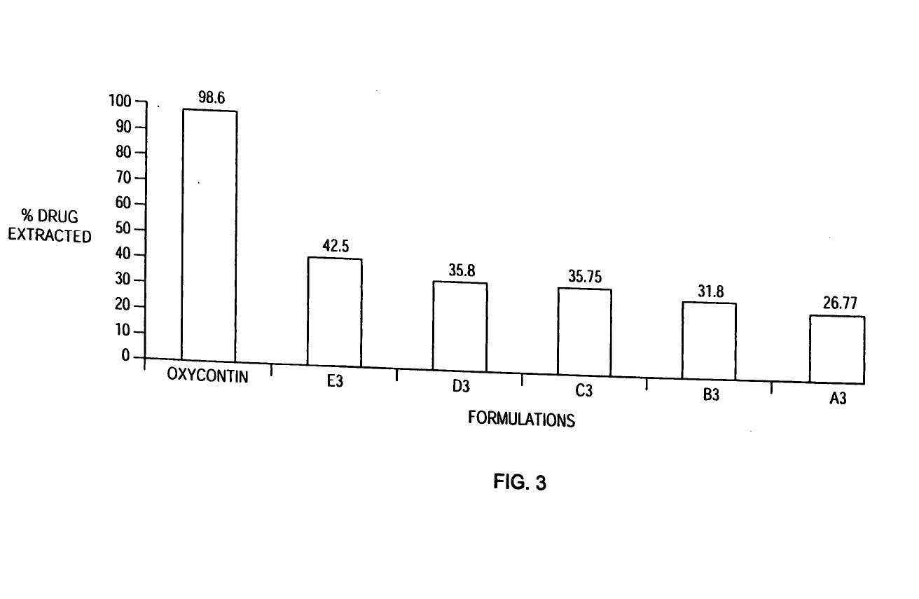

The present invention describes formulations which have abuse deterrent properties as described herein. Examples of specific oral solid dosage forms containing morphine, hydrocodone and oxycodone were evaluated using suitable analytical test methods, such as UV/VIS spectrophotometry. In the evaluation, dosage forms were crushed and contacted with a small amount of water (about a teaspoon or tablespoon). After attempting to dissolve the dosage form, the resultant material was drawn into a syringe, volume was measured and opioid content was quantitated. As shown in FIG. 1, almost 100% of the opioid can be extracted from standard formulations. Comparatively, as shown in FIG. 2, an abuse deterrent formulation of the present invention for the same opioids, provides a significantly lower percentage of extractable opioid. As shown in FIG. 1, approximately 93%, 103% and 99% of the opioid analgesic drugs contained in a dosage form were recoverable using the above described techniques. Comparatively, as shown in FIG. 2, using an abuse deterrent polymer of the present invention, only 9%, 5%, and 6% of the opioid analgesic drugs were recoverable.

In another embodiment, the present invention is a pharmaceutical composition that includes an opioid analgesic, one or more gel forming agents, and one or more mucous membrane irritants or nasal passageway tissue irritants. In another embodiment, the present invention includes a pharmaceutical composition, which includes an analgesic, one or more gel forming agents and one or more emetics as described herein. In another embodiment, the present invention includes a pharmaceutical composition, which includes an opioid analgesic, one or more mucous membrane irritants or nasal passageway tissue irritants and one or more emetics as described herein. In one particular embodiment, the present invention includes a pharmaceutical composition which includes an analgesic, one or more gel forming agents, one or more mucous membrane irritants and/or nasal passageway tissue irritants, and one or more emetics.

Each of the components (also referred to herein as “agents”) of the pharmaceutical composition, including classes of deterrents and constituents of each class of deterrent of the present invention, are described in more detail below. In certain embodiments, when administered in an amount which is less than the threshold amount for each particular agent, an agent included in the present invention can have no abuse deterrent effect or a beneficial effect upon an abuser, as described in more detail below.

A. Drugs Suitable for Use with the Present Invention

Any drug, therapeutically acceptable drug salt, drug derivative, drug analog, drug homologue, or polymorph can be used in the present invention. Suitable drugs for use with the present invention can be found in the Physician's Desk Reference, 59th Edition, the content of which is hereby incorporated by reference. In one embodiment, the drug is an orally administered drug. In certain embodiments, drugs susceptible to abuse are used. Drugs commonly susceptible to abuse include psychoactive drugs and analgesics, including but not limited to opioids, opiates, stimulants, tranquilizers, narcotics and drugs that can cause psychological and/or physical dependence. In one embodiment, the drug for use in the present invention can include amphetamines, norpseudoephedrine, amphetamine-like compounds, amphetamine and methamphetamine precursors including ephedrine, pseudoephedrine, and phenylpropanolamine, and methyl phenidate or combinations thereof. In another embodiment, the present invention can include any of the resolved isomers of the drugs described herein, and/or salts thereof.

A drug for use in the present invention which can be susceptible to abuse can be one or more of the following: acetaminophen, alfentanil, amphetamines, buprenorphine, butorphanol, carfentanil, codeine, dezocine, diacetylmorphine, dihydrocodeine, dihydromorphine, diphenoxylate, diprenorphine, etorphine, fentanyl, hydrocodone, hydromorphone, β-hydroxy-3-methylfentanyl, levo-α-acetylmethadol, levorphanol, lofentanil, meperidine, methadone, methylphenidate, morphine, nalbuphine, nalmefene, o-methylnaltrexone, naloxone, naltrexone, oxycodone, oxymorphone, pentazocine, pethidine, propoxyphene, remifentanil, sufentanil, tilidine, and tramodol, salts, derivatives, analogs, homologues, polymorphs thereof, and mixtures of any of the foregoing.

In another embodiment a drug for use with the present invention which can be susceptible to abuse includes one or more of the following: dextromethorphan (3-Methoxy-17-methyl-9a,13a, 14a-morphinan hydrobromide monohydrate), N-{1-[2-(4-ethyl-5-oxo-2-tetrazolin-1-yl)-ethyl]-4-methoxymethyl-4-piperidyl}propionanilide (alfentanil), 5,5-diallyl barbituric acid (allobarbital), allylprodine, alpha-prodine, 8-chloro-1-methyl-6-phenyl-4H-[1,2,4]triazolo[4,3-a][1,4]-benzodiazepine (alprazolam), 2-diethylaminopropiophenone (amfepramone), (±)-α-methyl phenethylamine (amphetamine), 2-(α-methylphenethyl-amino)-2-phenyl acetonitrile (amphetaminil), 5-ethyl-5-isopentyl barbituric acid (amobarbital), anileridine, apocodeine, 5,5-diethyl barbituric acid (barbital), benzylmorphine, bezitramide, 7-bromo-5-(2-pyridyl)-1H-1,4-benzodiazepin-2(3H)-one (bromazepam), 2-bromo-4-(2-chlorophenyl)-9-methyl-6H-thieno[3,2-f][1,2,4]-triazolo[4,3-a][1,4]diazepine (brotizolam), 17-cyclopropylmethyl-4,5α-epoxy-7a [(S)-1-hydroxy-1,2,2-trimethylpropyl]-6-methoxy-6,14-endo-ethanomorphinan-3-ol(buprenorphine), 5-butyl-5-ethyl barbituric acid (butobarbital), butorphanol, (7-chloro-1,3-dihydro-1-methyl-2-oxo-5-phenyl-2H-1,4-benzodiazepin-3-yl)-dimethyl carbamate (camazepam), (1S,2S)-2-amino-1-phenyl-1-propanol (cathine/D-norpseudoephedrine), 7-chloro-N-methyl-5-phenyl-3H-1,4-benzodiazepin-2-ylamine-4 oxide (chlordiazepoxide), 7-chloro-1-methyl-5-phenyl-1H-1,5-benzodiazepine-2,4(3H,5H)-dione (clobazam), 5-(2-chlorophenyl)-7-nitro-1H-1,4-benzodiazepin-2(3H)-one(clonazepam), clonitazene, 7-chloro-2,3-dihydro-2-oxo-5-phenyl-1H-1,4-benzodiazepine-3-carboxylic acid (clorazepate), 5-(2-chlorophenyl)-7-ethyl-1-methyl-1H-thieno[2,3-e][1,4]-diazepin-2(3H)-one (clotiazepam), 10-chloro-11b-(2-chlorophenyl)-2,3,7,11b-tetrahydrooxazolo[3,2-d][1,4]benzodiazepin-6(5H)-one(cloxazolam), (−)-methyl-[3β-benzoyloxy-2β(1αH,5αH)-tropane carboxylate (cocaine), 4,5α-epoxy-3-methoxy-17-methyl-7-morphinen-6α-ol (codeine), 5-(1-cyclohexenyl)-5-ethyl barbituric acid (cyclobarbital), cyclorphan, cyprenorphine, 7-chloro-5-(2-chlorophenyl)-1H-1,4-benzodiazepin-2(3H)-one (delorazepam), desomorphine, dextromoramide, (+)-(1-benzyl-3-dimethylamino-2-methyl-1-phenylpropyl)propionate (dextropropoxyphene), dezocine, diampromide, diamorphone, 7-chloro-1-methyl-5-phenyl-1H-1,4-benzodiazepin-2(3H)-one(diazepam), 4,5α-epoxy-3-methoxy-17-methyl-6α-morphinanol (dihydrocodeine), 4,5α-epoxy-17-methyl-3,6a-morphinandiol (dihydromorphine), dimenoxadol, dimephetamol[sic-Tr.Ed.], dimethyl thiambutene, dioxaphetyl butyrate, dipipanone, (6aR,10aR)-6,6,9-trimethyl-3-pentyl-6a,7,8,10a-tetrahydro-6H-benzo[c]chromen-1-ol (dronabinol), eptazocine, 8-chloro-6-phenyl-4H-[1,2,4]triazolo[4,3-a][1,4]benzodiazepine (estazolam), ethoheptazine, ethyl methyl thiambutene, ethyl-[7-chloro-5-(2-fluorophenyl)-2,3-dihydro-2-oxo-1H-1,4-benzodiazepin-3-carboxylate](ethyl loflazepate), 4,5α-epoxy-3-ethoxy-17-methyl-7-morphinen-6α-ol (ethylmorphine), etonitrazene, 4,5α-epoxy-7α-(1-hydroxy-1-methylbutyl)-6-methoxy-17-methyl-6,14-endo-etheno-morphinan-3-ol (etorphine), N-ethyl-3-phenyl-8,9,10-trinorbornan-2-ylamine (fencamfamine), 7-[2-(α-methylphenethylamino)-ethyl]theophylline (fenethylline), 3-(α-methylphenethylamino) propionitrile (fenproporex), N-(1-phenethyl-4-piperidyl) propionanilide (fentanyl), 7-chloro-5-(2-fluorophenyl)-1-methyl-1H-1,4-benzodiazepin-2(3H)-one (fludiazepam), 5-(2-fluorophenyl)-1-methyl-7-nitro-1H-1,4-benzodiazepin-2-(3H)-one (flunitrazepam), 7-chloro-1-(2-diethylaminoethyl)-5-(2-fluorophenyl)-1H-1,4-benzodiazepin-2(3H)-one (flurazepam), 7-chloro-5-phenyl-1-(2,2,2-trifluoroethyl)-1H-1,4-benzodiazepin-2(3H)-one (halazepam), 10-bromo-11b-(2-fluorophenyl)-2,3,7,11b-tetrahydro[1,3]oxazolo[3,2-d][1,4]benzodiazepin-6(5H)-one (haloxazolam), heroin, 4,5α-epoxy-3-methoxy-17-methyl-6-morphinanone (hydrocodone), 4,5α-epoxy-3-hydroxy-17-methyl-6-morphinanone (hydromorphone), hydroxypethidine, isomethadone, hydroxymethyl morphinan, 11-chloro-8,12b-dihydro-2,8-dimethyl-12b-phenyl-4H-[1,3]oxazino[3,2-d][1,4]benzodiazepin-4,7(6H)-dione (ketazolam), 1-[4-(3-hydroxyphenyl)-1-methyl-4-piperidyl]-1-propanone (ketobemidone), (3S,6S)-6-dimethylamino-4,4-diphenylheptan-3-yl acetate (levacetylmethadol (LAAM)), (−)-6-dimethylamino-4,4-diphenyl-3-heptanone (levomethadone), (+17-methyl-3-morphinanol (levorphanol), levophenacyl morphan, lofentanil, 6-(2-chlorophenyl)-2-(4-methyl-1-piperazinylmethylene)-8-nitro-2H-imidazo[1,2a][1,4]benzodiazepin-1(4H)-one (loprazolam), 7-chloro-5-(2-chlorophenyl)-3-hydroxy-1H-1,4-benzodiazepin-2(3H)-one (lorazepam), 7-chloro-5-(2-chlorophenyl)-3-hydroxy-1-methyl-1H-1,4-benzodiazepin-2(3H)-one (lormetazepam), 5-(4-chlorophenyl)-2,5-dihydro-3H-imidazo[2,1-a]isoindol-5-ol (mazindol), 7-chloro-2,3-dihydro-1-methyl-5-phenyl-1H-1,4-benzodiazepine (medazepam), N-(3-chloropropyl)-α-methylphenetylamine (mefenorex), meperidine, 2-methyl-2-propyl trimethylene dicarbamate (meprobamate), meptazinol, metazocine, methylmorphine, N,α-dimethylphenethylamine (methamphetamine), (±)-6-dimethylamino-4,4-diphenyl-3-heptanone (methadone), 2-methyl-3-o-tolyl-4(3H)-quinazolinone (methaqualone), methyl-[2-phenyl-2-(2-piperidyl)acetate] (methyl phenidate), 5-ethyl-1-methyl-5-phenyl barbituric acid (methyl phenobarbital), 3,3-diethyl-5-methyl-2,4-piperidinedione (methyprylon), metopon, 8-chloro-6-(2-fluorophenyl)-1-methyl-4H-imidazo[1,5-a][1,4]benzodiazepine (midazolam), 2-(benzhydrylsulfinyl) acetamide (modafinil), 4,5α-epoxy-17-methyl-7-morphinene-3,6α-diol (morphine), myrophine, (±)-trans-3-(1,1-dimethylheptyl)-7,8,10,10α-tetrahydro-1-hydroxy-6,6-dimethyl-6H-dibenzo[b,d]pyran-9(6αH)-one (nabilone), nalbuphen, nalorphine, narceine, nicomorphine, 1-methyl-7-nitro-5-phenyl-1H-1,4-benzodiazepin-2(3H)-one (nimetazepam), 7-nitro-5-phenyl-1H-1,4-benzodiazepin-2(3H)-one (nitrazepam), 7-chloro-5-phenyl-1H-1,4-benzodiazepin-2-(3H)-one (nordazepam), norlevorphanol, 6-dimethylamino-4,4-diphenyl-3-hexanone (normethadone), normorphine, norpipanone, the coagulated juice of the plants belonging to the species Papaver somniferum (opium), 7-chloro-3-hydroxy-5-phenyl-1H-1,4-benzodiazepin-2-(3H)-one (oxazepam), (cis-trans)-10-chloro-2,3,7,11b-tetrahydro-2-methyl-11b-phenyloxazolo[3,2-d][1,4]benzodiazepin-6-(5H)-one (oxazolam), 4,5α-epoxy-14-hydroxy-3-methoxy-17-methyl-6-morphinanone (oxycodone), oxymorphone, plants and plant parts of the plants belonging to the species Papaver somniferum (including the subspecies setigerum) (Papaver somniferum), papaveretum, 2-imino-5-phenyl-4-oxazolidinone (pemoline), 1,2,3,4,5,6-hexahydro-6,11-dimethyl-3-(3-methyl-2-butenyl)-2,6-methano-3-benzazocin-8-ol (pentazocine), 5-ethyl-5-(1-methylbutyl) barbituric acid (pentobarbital), ethyl-(1-methyl-4-phenyl-4-piperidine-carboxylate) (pethidine), phenadoxone, phenomorphan, phenazocine, phenoperidine, piminodine, pholcodeine, 3-methyl-2-phenyl morpholine (phenmetrazine), 5-ethyl-5-phenyl barbituric acid (phenobarbital), α,α-dimethyl phenethylamine (phentermine), 7-chloro-5-phenyl-1-(2-propinyl)-1H-1,4-benzodiazepin-2(3H)-one (pinazepam), α-(2-piperidyl)benzhydryl alcohol (pipradol), 1′-(3-cyano-3,3-diphenylpropyl)[1,4′-bipiperidine]-4′-carboxamide (piritramide), 7-chloro-1-(cyclopropylmethyl)-5-phenyl-1H-1,4-benzodiazepin-2(3H)-one (prazepam), profadol, proheptazine, promedol, properidine, propoxyphene, N-(1-methyl-2-piperidinoethyl)-N-(2-pyridyl) propionamide, methyl-{3-[4-methoxycarbonyl-4-(N-phenylpropaneamido)piperidino]propanoate} (remifentanil), 5-sec.-butyl-5-ethyl barbituric acid (secbutabarbital), 5-allyl-5-(1-methylbutyl) barbituric acid (secobarbital), N-{4-methoxymethyl-1-[2-(2-thienyl)ethyl]-4-piperidyl}propionanilide (sufentanil), 7-chloro-2-hydroxy-methyl-5-phenyl-1H-1,4-benzodiazepin-2-(3H)-one (temazepam), 7-chloro-5-(1-cyclohexenyl)-1-methyl-1H-1,4-benzodiazepin-2(3H)-one (tetrazepam), ethyl-(2-dimethylamino-1-phenyl-3-cyclohexane-1-carboxylate) (tilidine (cis and trans)), tramadol, 8-chloro-6-(2-chlorophenyl)-1-methyl-4H-[1,2,4]triazolo[4,3-a][1,4]benzodiazepine (triazolam), 5-(1-methylbutyl)-5-vinyl barbituric acid (vinylbital), (1R*,2R*)-3-(3-dimethylamino-1-ethyl-2-methyl-propyl) phenol, (1R,2R,4S)-2-[dimethylamino)methyl-4-(p-fluorobenzyloxy)-1-(m-methoxyphenyl) cyclohexanol, each optionally in the form of corresponding stereoisomeric compounds as well as corresponding derivatives, especially esters or ethers, and all being physiologically compatible compounds, especially salts and solvates.

In one embodiment, a pharmaceutical composition of the present invention includes one or more opioids such as hydrocodone, morphine and oxycodone and/or salts thereof, as the therapeutically active ingredient. Typically when processed into a suitable dosage form, as described in more detail below, the drug can be present in such dosage forms in an amount normally prescribed, typically about 0.5 to about 25 percent on a dry weight basis, based on the total weight of the formulation.

With respect to analgesics in unit dose form, such an amount can be typically from about 5, 25, 50, 75, 100, 125, 150, 175 or 200 mg. More typically, the drug can be present in an amount from 5 to 500 mg or even 5 to 200 mg. In other embodiments, a dosage form contains an appropriate amount of drug to provide a therapeutic effect.

In another embodiment, the present invention includes one or more drugs which are not typically susceptible to abuse in addition to a drug which is susceptible to abuse, described above. In certain embodiments, the one or more additional drugs which are not typically susceptible to abuse can have an abuse deterrent effect (as described in more detail below) when administered in combination with a drug which is susceptible to abuse. In one embodiment of a dosage form of the present invention which includes a drug that is susceptible to abuse, the one or more additional drugs which can induce an abuse deterrent effect can be included in the dosage form in a sub-therapeutic or sub-clinical amount.

As used herein, “sub-therapeutic” or “sub-clinical” refer to an amount of a referenced substance that if consumed or otherwise administered, is insufficient to induce an abuse deterrent effect (e.g., nausea) in an average subject or is insufficient to meet or exceed the threshold dose necessary for inducing an abuse deterrent effect.

Accordingly, when an embodiment of a dosage form of the present invention is administered in accordance with a health care provider prescribed dosage and/or manner, the one or more additional drugs which can induce an abuse deterrent effect will not be administered in an amount sufficient to induce an abuse deterrent effect. However, when a certain embodiment of the present invention is administered in a dose and/or manner that is different from a health care provider prescribed dose, (i.e., the drug is abused or the dosage form is tampered with) the content of a formulation which can cause an abuse deterrent effect according to the present invention will be sufficient to induce an abuse deterrent effect. Suitable examples of drugs which can be administered in sub-therapeutic amounts in the present invention include niacin, atropine sulfate, homatropine methylbromide, sildenafil citrate, nifedipine, zinc sulfate, dioctyl sodium sulfosuccinate and capsaicin.

B. Viscosity Increasing/Gel Forming Agents

As described above, the present invention can include one or more viscosity increasing or gel forming agents (hereafter referred to as gel forming agents). The total amount of gel forming agent is typically about 3 to about 70 percent, preferably about 3 to about 40 percent, on a dry weight basis of the composition.

Suitable gel forming agents include compounds that, upon contact with a solvent (e.g., water), absorb the solvent and swell, thereby forming a viscous or semi-viscous substance that significantly reduces and/or minimizes the amount of free solvent which can contain an amount of solubilized drug, and which can be drawn into a syringe. The viscous or gelled material can also reduce the overall amount of drug extractable with the solvent by entrapping the drug in a gel matrix. In one embodiment, typical gel forming agents include pharmaceutically acceptable polymers, typically hydrophilic polymers, such as hydrogels.

In some embodiments, the polymers exhibit a high degree of viscosity upon contact with a suitable solvent. The high viscosity can enhance the formation of highly viscous gels when attempts are made by an abuser to crush and dissolve the contents of a dosage form in an aqueous vehicle and inject it intravenously.

More specifically, in certain embodiments the polymeric material in the present invention forms a viscous or gelled material upon tampering. In such embodiments, when an abuser crushes and dissolves the dosage form in a solvent (e.g., water or saline), a viscous or semi-viscous gel is formed. The increase in the viscosity of the solution discourages the abuser from injecting the gel intravenously or intramuscularly by preventing the abuser from transferring sufficient amounts of the solution to a syringe to cause a desired “high” once injected. The increase in viscosity of the solution also discourages the abuser from inhaling (e.g., nasal or oral inhalation of the gelled material). In another embodiment, the increase in viscosity of the solution discourages the use of legitimate, over the counter, and/or prescription drugs that are included in embodiments of the present invention in the illicit manufacture of other drugs. Specifically, the gel restricts the solubilization of the drug prior to the conversion of the drug to another drug, e.g., the illicit use of pseudoephedrine in the manufacture of methamphetamine.

In one embodiment, suitable polymers include one or more pharmaceutically acceptable polymers selected from any pharmaceutical polymer that will undergo an increase in viscosity upon contact with a solvent, e.g., as described in U.S. Pat. No. 4,070,494, the entire content of which is hereby incorporated by reference. Preferred polymers can include alginic acid, polyacrylic acid, karaya gum, tragacanth, polyethylene oxide, polyvinyl alcohol, and methyl cellulose including sodium carboxy methyl cellulose, hydroxyethyl methyl cellulose hydroxypropyl methyl cellulose and carbomers. In preferred embodiments, the polymers include:

a) Polyethylene Oxide
In some embodiments, the polymer includes polyethylene oxide. In certain embodiments, the polyethylene oxide can have an average molecular weight ranging from about 300,000 to about 5,000,000, more preferably from about 600,000 to about 5,000,000, and most preferably at least about 5,000,000. In one embodiment, the polyethylene oxide includes a high molecular weight polyethylene oxide.
In one embodiment, the average particle size of the polyethylene oxide ranges from about 840 to about 2,000 microns. In another embodiment, the density of the polyethylene oxide can range from about 1.15 to about 1.26 g/ml. In another embodiment, the viscosity can range from about 8,800 to about 17,600 cps.
The polyethylene oxide used in a directly compressible formulation of the present invention is preferably a homopolymer having repeating oxyethylene groups, i.e., —(—O—CH₂—CH₂—)ₙ—, where n can range from about 2,000 to about 180,000. Preferably, the polyethylene oxide is a commercially available and pharmaceutically acceptable homopolymer having moisture content of no greater than about 1% by weight. Examples of suitable, commercially available polyethylene oxide polymers include Polyox®, WSRN-1105 and/or WSR-coagulant, available from Dow chemicals. In another embodiment, the polymer can be a copolymer, such as a block copolymer of PEO and PPO.
In some embodiments, the polyethylene oxide powdered polymers can contribute to a consistent particle size in a directly compressible formulation and eliminate the problems of lack of content uniformity and possible segregation.

b) Polyvinyl Alcohol
In one embodiment, the gel forming agent includes polyvinyl alcohol. In one embodiment, the polyvinyl alcohol can have a molecular weight ranging from about 20,000 to about 200,000. In one embodiment, the specific gravity of the polyvinyl alcohol can range from about 1.19 to about 1.31 and the viscosity from about 4 to about 65 cps. The polyvinyl alcohol used in the formulation is preferably a water-soluble synthetic polymer represented by —(—C₂H₄O—)ₙ—, where n can range from about 500 to about 5,000. Examples of suitable, commercially available polyvinyl alcohol polymers include PVA, USP, available from Spectrum Chemical Manufacturing Corporation, New Brunswick, N.J. 08901.

c) Hydroxypropyl Methyl Cellulose
In one embodiment, the gel forming agent includes hydroxypropyl methyl cellulose (Hypromellose). In certain embodiments, the hydroxypropyl methyl cellulose can have a molecular weight ranging from about 10,000 to about 1,500,000. In one embodiment, the hydroxypropyl methyl cellulose has a molecular weight from about 5000 to about 10,000, i.e., a low molecular weight hydroxypropyl methyl cellulose polymer. In one embodiment, the specific gravity of the hydroxypropyl methyl cellulose can range from about 1.19 to about 1.31, with an average specific gravity of about 1.26 and a viscosity of about 3600 to 5600. The hydroxypropyl methyl cellulose used in the formulation can be a water-soluble synthetic polymer. Examples of suitable, commercially available hydroxypropyl methylcellulose polymers include Methocel K100 LV and Methocel K4M, available from Dow chemicals.

d) Carbomers
In one embodiment, the present invention includes carbomers. In one embodiment, the carbomers can have a molecular weight ranging from 700,000 to about 4,000,000,000. In one embodiment, the viscosity of the polymer can range from about 4000 to about 39,400 cps. Examples of suitable, commercially available carbomers include polyacrylic acids such as carbopol 934P NF, carbopol 974P NF and carbopol 971P NF, available from Noveon Pharmaceuticals.

Following the teachings set forth herein, other suitable gel forming agents can include one or more of the following polymers: ethyl cellulose, cellulose acetate, cellulose acetate propionate, cellulose acetate butyrate, cellulose acetate phthalate and cellulose triacetate, cellulose ether, cellulose ester, cellulose ester ether, and cellulose, acrylic resins comprising copolymers synthesized from acrylic and methacrylic acid esters, the acrylic polymer may be selected from the group consisting of acrylic acid and methacrylic acid copolymers, methyl methacrylate copolymers, ethoxyetlryl methacrylates, cyanoetlryl methacrylate, poly(acrylic acid), poly(methaerylic acid), methacrylic acid alkylamide copolymer, poly(methyl methacrylate), polymethacrylate, poly(methyl methacrylate) copolymer, polyacrylamide, aminoalkyl methacrylate copolymer, poly(methacrylic acid anhydride), and glycidyl methacrylate copolymers.

Any of the above described polymers can be combined together or combined with other suitable polymers, and such combinations are within the scope of the present invention.

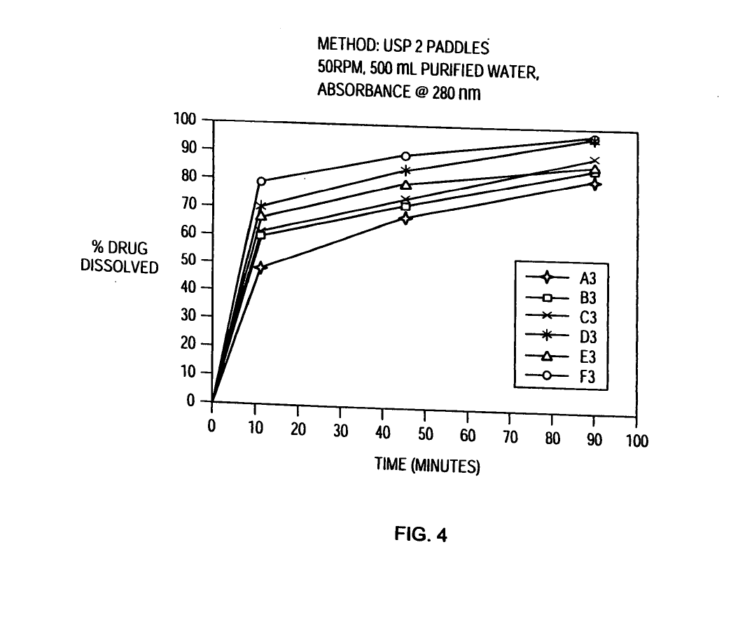

In one embodiment, the abuse deterrent, gel forming agent can prevent less than or equal to about 95%, 94%, 70%, 60%, 54%, 50%, 45%, 40%, 36%, 32%, 30%, 27%, 20%, 10%, 9%, 6%, 5% or 2% of the total amount of a pharmaceutical susceptible to abuse in a dosage form from being recovered from a solvent in contact with a dosage form of the present invention. As shown in FIG. 3, formulations A3, B3, C3, D3 and E3 reduce the amount of drug extractable or recoverable from a dosage form of the present invention which includes a gel forming agent of the present invention. Specifically, formulation A3 provides for recovery of 26.77% of the total amount of drug in the dosage form, formulation B3 provides for recovery of 31.8% of the total amount of drug in the dosage form, formulation C3 provides for recovery of 35.75% of the total amount of drug in the dosage form, formulation D3 provides for recovery of 35.8% of the total amount of drug in the dosage form, and formulation E3 provides for recovery of 42.5% of the total amount of drug in the dosage form. In FIG. 3, all five formulations A3 through E3 are compared with a standard dosage form of oxycontin, which provided for recovery of 98.6% of the total amount of drug in the dosage form.

The five formulations A3 through E3 are set forth in Examples 14 through 18, respectively.

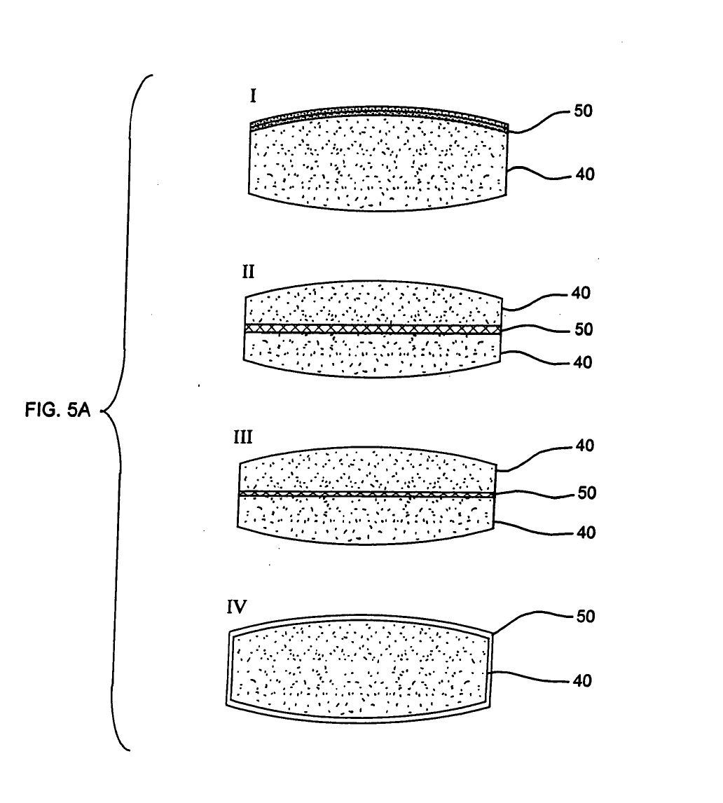

It should be noted that the above described formulations also have dissolution profiles as determined by the USP 2-paddle method, as shown in FIG. 4. In particular, for formulations A3 through E3, about 50% to about 82% of each formulation dissolves after about 15 minutes and about 80% to about 95% dissolves after 90 minutes. FIG. 4 further includes the dissolution profile of formulation F3. With respect to FIG. 4, the composition of formulation F3 is set forth in Example 19.

The above described gel forming agents can be further optimized as necessary or desired in terms of viscosity, molecular weight, etc.

In another embodiment, the polymer can be selected such that the polymer can reduce or prevent abuse or misuse of a drug via nasal inhalation (snorting). In one such embodiment, a portion of a crushed dosage form can be inhaled and thereby contact the nasal mucosa. In one embodiment, about 30% to 60% (by weight) of a crushed and subsequently inhaled dosage form of the present invention remains in contact with the nasal mucosa of the nasal cavity.

The polymer (e.g., polyox) included in the crushed dosage form of the present invention then reacts with liquid (e.g., water in the mucous) on the nasal mucosa, forming a viscous gel. Once the gel forms on the nasal mucosa, only about 5% to 15%, more typically about 10% of the drug in the gel remains available for absorption through the mucosa, thereby significantly reducing the occurrence of a drug “high.”

A comparison of the amount of drug extractable from commercially available dosage forms to a dosage form of the present invention is provided in the following drug extraction test table:

Extraction Data Table

| | | Embodiment of the Present |
|---|---|---|
| | | Invention as Shown in |
| Mallinckrodt 5 mg | | Example 44: 5 mg |
| Oxycodone HCl Tablet | Oxycontin ® 40 mg | Oxycodone HCl |
| Test A - 81.3% | Test A - 98.6% | Test A - 14.4% |
| Test B - 86.4% | Test B - 100.5% | Test B - 7.7% |
| Test C - 85.1% | Test C - 98.7% | Test C - 9.2% |
| Test D - 87.8% | Test D - 99.3% | Test D - 7.7% |
| Average = 85.1% | Average = 99.3% | Average = 9.8% |

Furthermore, the gel formed in the nasal cavity can cause one or more of acute sinusitis or chronic sinusitis, and/or cause blockage of one or more of the sphenoid, maxillary, ethmoid and frontal sinuses, and/or complicate (e.g., inhibit) the uncinate process and the ostio-meatal complex. Additionally, in certain embodiments, the gel can block the interior nasal valve, thus significantly restricting airflow, and thereby reducing or preventing abuse or misuse of a dosage form of the present invention. The reduction in airflow can also impair the senses of smell and taste of the abuser.

In another embodiment, the gel which is adhered to the nasal mucosa inhibits the mucociliary clearance system. Typically the mucociliary clearance system in a healthy adult produces about 800 ml. to about 1200 ml. of fluid per day in order to maintain clear nasal passages. By inhaling a dosage form of the present invention, at least 50%, 60%, 75%, 80%, 85%, 90%, more typically 95% of the gel which is adhered to the nasal mucosa can be cleared in 1 to 5 days, through normal mucociliary clearance. In one embodiment of the invention, an above described percentage of the gel can be cleared in greater than about 1 day. Thus, in the present invention the undesirable sinus related effects described above can last for 1 or more days and accordingly once a dosage form of the present invention is abused, the abuse deterrent effects can reduce or prevent inhalation or snorting abuse or misuse of a dosage form of the present invention, as well as other dosage forms which do not cause an abuse deterrent effect, for an extended period of time.

The formation of the gel in the nasal passages can also prevent nose blowing and other attempts (e.g., washing with a saline solution) to clear the gel from the nasal mucosa.

In certain embodiments, the methods and compositions directed to polymers for reducing or preventing abuse or misuse of a drug via nasal inhalation can be combined with one or more suitable irritants or other abuse deterrents described herein to further reduce or prevent the abuse or misuse of a drug included in a dosage form of the present invention, as described below.

C. Mucous Membrane Irritants and/or Respiratory Passageway Tissue Irritants

As described above, the present invention can include one or more mucous membrane irritants, and/or respiratory passageway (e.g., oral or nasal) tissue irritants, and/or irritants to oral cavity or throat including the pharynx. In one embodiment, suitable mucous membrane irritants and/or respiratory (e.g., oral or nasal) passageway tissue irritants include compounds that are generally considered pharmaceutically inactive, yet can induce irritation. Such compounds include, but are not limited to surfactants, including in certain embodiments anionic surfactants as described herein below. In one embodiment, suitable surfactants include sodium lauryl sulfate, poloxamer, sorbitan monoesters and glyceryl monooleates. Other suitable compounds are believed to be within the knowledge of a practitioner skilled in the relevant art, and include certain vasodilators such as nicotinic acid, and can be found in the Handbook of Pharmaceutical Excipients, 4th Ed. (2003), the entire content of which is hereby incorporated by reference.

In another embodiment, the irritant can be pharmaceutically active. In such embodiments, the irritant can include one or more members of the vanilloid family and derivatives thereof, including capsaicin.

Examples of suitable irritants may be of natural or synthetic origin and include mustard, for example, allyl isothiocyaanate and p-hydroxybenzyl isothiocyanate; capsaicinoids such as capsaicin, dihydrocapsaicin, nordihydrocapsaiscin, homocapsaicin, and homodihydrocapsaicin, mint; aspirin; and acids such as acids with one or more carboxyl moieties such as formic acid, acetic acid, propionic acidy, butyric acid, valeric acid, caproic acid, caprillic acid, capric acid, oxalic acid, malonic acid, succicnic acid, glutaric acid, adipic acid, maleic acid, fumaric acid, and citric acid. Preferred local irritants for use in the present invention are capsaicinoids such as, for example, capsaicin.

In one embodiment of the present invention, the irritant can be present in an amount of from 1 to 20 percent by weight on a solid basis, preferably 1 to 10 percent by weight on a solid basis. In another embodiment, the amount of irritant can be present in an amount of 5 to 15 percent by weight. In another embodiment, the irritant can be present in an amount of at least 5 percent by weight. In yet another embodiment, the irritant can be present in an amount from 1 to 5 percent by weight. In another embodiment, the amount of irritant can be present in an amount from 1 to 3 percent by weight.

In certain embodiments, the irritant can deter abuse of a dosage form when a potential abuser tampers with a dosage form of the present invention. Specifically, in such embodiments, when an abuser crushes the dosage form, the irritant is exposed. The irritant discourages inhalation (e.g., oral or nasal) of the crushed dosage form by inducing pain and/or irritation of the abuser's mucous membrane and/or respiratory passageway tissue. In one embodiment, the irritant discourages inhalation (e.g., via breathing through the mouth or via snorting through the nose) by inducing pain and/or irritation of the abuser's respiratory (e.g., nasal or oral) passageway tissue.

In one embodiment, the present invention includes one or more mucous membrane irritants to cause irritation of mucous membranes located anywhere on or in the body, including membranes of the mouth, eyes, nose and intestinal tract. Such compositions can deter abuse via oral, intra-ocular, rectal, or vaginal routes.

The above-described irritants can be further optimized as necessary or desired in terms of concentration, irritation severity, etc.

In one embodiment, the surfactant can be an anionic surfactant. In one such embodiment, the anionic surfactant (e.g., docusate) can also function as a potential laxative and/or stool softener at excess doses. In one embodiment, the surfactant can be sodium and/or calcium and/or potassium dioctyl sulfosuccinate, as described further below.

In certain embodiments, the present invention includes one or more surfactants set forth by general functionality, application, class or family in the following list:

| | |
|---|---|
| SURFACTANT DESCRIPTIONS | REPRESENTATIVE MATERIALS |

By Functionality

| | |
|---|---|
| Anionic | |
| Cationic | |
| Nonionic | |
| Amphoteric (Zwitterionic) | Cocamidopropyl betaine |

By Application

Foaming agents
Anti-foaming agents
Dispersants
Emulsifiers
Solubilizers
Emollients
Gel enhancers
Detergents
Chelating agents
Wetting agents

By Chemical Family

| | |
|---|---|
| Ethylene oxide/propylene oxide copolymers | Tergitol XD |
| Polyoxyethylene/polyoxypropylene | Poloxamers (188, 237, 338, 407) |
| copolymers | |
| Polyoxyethylene/polypropylene copolymers | |
| Polyoxyethylene/polybutylene copolymers | |
| Perfluoropolyether ammonium carboxylate | |
| Sulfates | |
| Alkyl sulfates | |
| Lauryl sulfates | SLS, Ammonium lauryl sulfate |
| Alkyl sulfate salts | Sodium ethylhexyl sulfate |
| Alkyl ether sulfates | Ammonium laureth sulfate, sodium |
| | laureth sulfate |
| Alkaryl ether sulfates | |
| Sulfated fats and oils | |
| Sulfated monoglycerides | |
| Sulfated alkanolamides | |
| Sulfonates | |
| Olefin sulfonates | |
| Alkylaryl sulfonates | Triton X-200 |
| Polyether sulfonates | |
| Naphthalene sulfonates | |
| Phosphates | |
| Phosphate esters | Triton QS |
| Alkyl phosphates | Stearyl phosphate |
| Alkyl phosphate salts | |
| Alkyl ether phosphates | Trilaureth phosphate |
| Ammonium compounds | |
| Alkyl ammonium salts | |
| Alkyl trimethylammonium salts | |
| Quaternary ammonium chlorides | Benzalkonium chloride |
| Amines and Amides | |
| Alkyl amines | Triethanolamine |
| Amine oxides | Oleamine oxide, Lauramine oxide, |
| | Stearamine oxide |
| Ethoxylated amines | Polyoxyethylene octadecylamine |
| Alkyl aminopropionates | |
| Alkanolamides | |
| Diethanolamides | Lauramide DEA, Cocamide DEA, |
| | Oleamide DEA, Soyamide DEA |
| Monoisopropanolamides | Cocamide MIPA, Lauramide MIPA |
| Monoethanolamides | Acetamide MEA, Cocamide MEA, |
| | Lauramide MEA, Stearamide MEA |
| Amido amines | Cocamidopropyl dimethylamine, |
| | Stearamidopropyl dimethylamine |
| Silicones and silicates | |
| Dimethicones | |
| Simethicone | |
| Silicates | Sodium silicate, Sodium metasilicate |
| Sarcosines | |
| Lauryl sarcosines | |
| Cocoyl sarcosines | |
| Alcohols | |
| Ethoxylated alcohols | Laureth series, Steareth series, Ceteth |
| | series, Oleth series, Tergitol TMN |
| Propoxylated alcohols | |
| Alkyl alcohols and blends | Cetyl alcohol, Stearyl alcohol, Caprylic |
| | alcohol, Lauryl alcohol, Myristyl alcohol |
| Glycols | Nonoxynol |
| Polyethylene glycols | PEG |
| Acetylenic alcohol | 3-methyl-1-butyn-3-ol |
| Ethers and ethoxylates | |
| Glycol ethers | Triethylene glycol monooctyl ether |
| Ethoxylated glycerine | Glycereth-12 |
| Polyoxyethylene alkyl ethers | Polyoxyl 20 cetostearyl ether, Polyoxyl |
| | 2 cetyl ether |
| Lanolin ethoxylates | |
| Octyl phenol ethoxylates | Triton X |
| Nonyl phenol ethoxylates | |
| Oleic acid ethoxylates | |
| Cocoamine ethoxylates | |
| Tallow amine ethoxylates | |
| Stearic acid ethoxylates | |
| Fatty acid ethoxylates | |
| Ethoxylated sorbitan esters | Polysorbates |
| Esters | |
| Methyl esters and blends | Methyl laurate, Methyl palmitate, Methyl |
| | decanoate, Methyl soyate |
| Sobitan esters | Sorbitan stearate, Sorbitan oleate, |
| | Sorbitan laurate |
| Sucrose esters | |
| Fatty acid esters | |
| Polyethylene glycol esters | PEG-8 oleate, PEG-8 stearate |
| Alcohol esters | |
| Glycerol esters | Glyceryl oleate, Glyceryl stearate, |
| | Glyceryl Laurate |
| Glycol esters | Propylene glycol monostearate, Glycol |
| | distearate |
| Butyl and isopropyl esters | Butyl stearate, Isopropyl palmitate, |
| | Isopropyl myristate |
| Sulfosuccinate esters | |
| Sulfuric acid esters | |
| Alkyl carboxylates | |
| Alkyl ether carboxylates | |
| Lactylates | |
| Glutamates | |
| Sarcosinates | Lauroyl sarcosinates, Cocoyl |
| | sarcosinates |
| Organic acids | |
| Carboxylic acids | Stearic acid, Palmitic acid, Lauric acid, |
| | Myristic acid |
| Fatty acids | Oleic acid, Palm kernel fatty acid, |
| | Coconut fatty acid, Erucic acid |
| Plant oils | Canola oil, Palm oil, Coconut oil, |
| | Apricot kernel oil, Castor oil |
| Sulfosuccinates | Dioctyl sodium sulfosuccinate, Dioctyl |
| | calcium sulfosuccinate |
| Taurate salts | Sodium methyl cocoyl taurate, Sodium |
| | methyl oleoyl taurate |
| EDTAs | Disodium EDTA |
| Alkyl iminodipropionates | |
| Alkyl imidazoline derivatives | |
| Alkyl pyridinium salts | |
| Lanolin derivatives | Acetol, Lantrol |
| Protein derivatives | Hydrolyzed wheat gluten, Hydrolyzed |
| | collagen |
| Alkyl polyglycosides | Glucopon, Isodecyloxypropyamine, |
| | Lauryl glucoside |
| Alkyl oligosaccharides | |
| Cyclodextrins | |
| Lecithins | |
| Betaines | Cocobetaine, Cocoamido propyl |
| | betaine |
| Sultaines | Cocoamidopropyl sultaine |

In another embodiment, the present invention includes one or more non-surfactant type irritants.

In one embodiment, the present invention includes a slight irritant, which means that as part of good industrial and personal hygiene and safety procedure, one should avoid all unnecessary exposure to the chemical substance and ensure prompt removal from skin, eyes and clothing. Inhalation of high concentrations of dust may cause coughing and sneezing, while ingestion of extremely large oral doses may cause gastrointestinal disturbances. Further, while no adverse effects are expected upon contact with skin or upon chronic exposure or for aggravating a pre-existing condition, eye contact may cause mild irritation, redness and/or pain.

In yet another embodiment, the present invention includes a moderate irritant, which means that the substance may be may be harmful if swallowed or inhaled. The substance may cause irritation to skin, eyes, and respiratory tract, including shortness of breath. Furthermore, large oral doses may cause irritation to the gastrointestinal tract.

In yet another embodiment, the present invention includes a severe or toxic irritant, which means that the inhalation of vapors can cause coughing, choking, inflammation of the nose, throat, and upper respiratory tract, and in severe cases, pulmonary edema, circulatory failure, and death. Further, ingestion may cause immediate pain and/or burns of the mouth, throat, esophagus and gastrointestinal tract and may cause nausea, vomiting, and diarrhea. Additionally, skin contact can cause redness, pain, and/or severe skin burns. Also, contact may cause severe burns and permanent eye damage.

In another embodiment, the present invention includes one or more of the following irritants having the associated irritation level classification:

| | Compound | Classification |
|---|---|---|
| 1. | ACETYL TRIBUTYL CITRATE | Slight |
| 2. | ALPHA-TOCOPHEROL | Slight |
| 3. | ALUMINUM MAGNESIUM SILICATE | Slight |
| 4. | ALUMINUM SILICATE | Slight |
| 5. | ASCORBIC ACID | Slight |
| 6. | ASCORBYL PALMITATE | Slight |
| 7. | BENTONITE | Slight |
| 8. | CALCIUM ASCORBATE | Slight |
| 9. | CALCIUM CITRATE | Slight |
| 10. | CALCIUM SILICATE | Slight |
| 11. | CARBOMER | Slight |
| 12. | ESSENCE ORANGE | Slight |
| 13. | ETHYLCELLULOSE | Slight |
| 14. | FERRIC OXIDE | Slight |
| 15. | FERROSOFERRIC OXIDE | Slight |
| 16. | FERROUS FUMARATE | Slight |
| 17. | FERROUS OXIDE | Slight |
| 18. | GLYCERIN | Slight |
| 19. | HYDROXYPROPYL CELLULOSE | Slight |
| 20. | HYDROXYPROPYL METHYLCELLULOSE | Slight |
| | PHTHALATE | |
| 21. | KAOLIN | Slight |
| 22. | KOLA NUT EXTRACT | Slight |
| 23. | LACTOSE | Slight |
| 24. | LEUCINE | Slight |
| 25. | LOCUST BEAN GUM | Slight |
| 26. | MAGNASWEET 135 | Slight |
| 27. | MAGNESIUM ACETATE | Slight |
| 28. | MAGNESIUM CARBONATE | Slight |
| 29. | MAGNESIUM SILICATE | Slight |
| 30. | MAGNESIUM TRISILICATE | Slight |
| 31. | MALTODEXTRIN | Slight |
| 32. | MEGLUMINE | Slight |
| 33. | METHYLCELLULOSE | Slight |
| 34. | MINERAL OIL | Slight |
| 35. | ORANGE OIL | Slight |
| 36. | PEANUT OIL | Slight |
| 37. | POTASSIUM BICARBONATE | Slight |
| 38. | POTASSIUM CARBONATE | Slight |
| 39. | POTASSIUM CHLORIDE | Slight |
| 40. | PROPYL GALLATE | Slight |
| 41. | SILICON | Slight |
| 42. | SILICONE | Slight |
| 43. | SODIUM BICARBONATE | Slight |
| 44. | SODIUM STARCH GLYCOLATE | Slight |
| 45. | SODIUM SUCCINATE | Slight |
| 46. | SODIUM SULFATE | Slight |
| 47. | SODIUM SULFITE | Slight |
| 48. | SODIUM THIOSULFATE | Slight |
| 49. | SOYBEAN OIL | Slight |
| 50. | SPEARMINT | Slight |
| 51. | SPEARMINT OIL | Slight |
| 52. | TAGATOSE, D | Slight |
| 53. | TALC | Slight |
| 54. | TRIACETIN | Slight |
| 55. | VANILLIN | Slight |
| 56. | XANTHAN GUM | Slight |
| 57. | ZEIN | Slight |
| 58. | ACESULFAME POTASSIUM | Moderate |
| 59. | ALUMINUM HYDROXIDE | Moderate |
| 60. | AMMONIUM CALCIUM ALGINATE | Moderate |
| 61. | AMMONIUM PHOSPHATE | Moderate |
| 62. | AMYL ACETATE | Moderate |
| 63. | ANETHOLE | Moderate |
| 64. | ASPARTAME | Moderate |
| 65. | BENZYL BENZOATE | Moderate |
| 66. | BUTYLATED HYDROXYTOLUENE | Moderate |
| 67. | CALCIUM ACETATE | Moderate |
| 68. | CALCIUM PHOSPHATE | Moderate |
| 69. | CALCIUM SULFATE | Moderate |
| 70. | CINNAMALDEHYDE | Moderate |
| 71. | CINNAMON OIL | Moderate |
| 72. | DIBUTYL PHTHALATE | Moderate |
| 73. | EDETATE CALCIUM DISODIUM | Moderate |
| 74. | EDETATE DISODIUM | Moderate |
| 75. | ETHYLPARABEN | Moderate |
| 76. | GUM ROSIN | Moderate |
| 77. | LEVOMENTHOL | Moderate |
| 78. | MAGNESIIUM SULFATE, ANHYDROUS | Moderate |
| 79. | MALTOL | Moderate |
| 80. | MENTHOL | Moderate |
| 81. | METHIONINE | Moderate |
| 82. | METHYL ETHYL KETONE | Moderate |
| 83. | METHYL SALICYLATE | Moderate |
| 84. | METHYLPARABEN | Moderate |
| 85. | PEPPERMINT OIL | Moderate |
| 86. | POTASSIUM HYDROXIDE | Moderate |
| 87. | PROPYLPARABEN | Moderate |
| 88. | PROPYLPARABEN SODIUM | Moderate |
| 89. | SACCHARINE | Moderate |
| 90. | SIMETHICONE | Moderate |
| 91. | SODIUM ACETATE | Moderate |
| 92. | SODIUM ALGINATE | Moderate |
| 93. | SODIUM BENZOATE | Moderate |
| 94. | SODIUM CARBONATE | Moderate |
| 95. | SODIUM CASEINATE | Moderate |
| 96. | SODIUM CHLORIDE | Moderate |
| 97. | SODIUM CITRATE | Moderate |
| 98. | SODIUM HYDROXIDE | Moderate |
| 99. | SODIUM PHOSPHATE | Moderate |
| 100. | SODIUM PROPIONATE | Moderate |
| 101. | SORBIC ACID | Moderate |
| 102. | SUCCINIC ACID | Moderate |
| 103. | TRIETHYL CITRATE | Moderate |
| 104. | TRIMYRISTIN | Moderate |
| 105. | TROMETHAMINE | Moderate |
| 106. | ZINC SULFATE | Moderate |
| 107. | ACETIC ACID | Severe |
| 108. | ACETIC ANHYDRIDE | Severe |
| 109. | ALGINIC ACID | Severe |
| 110. | BENZOIC ACID | Severe |
| 111. | BISMUTH SUBCARBONATE | Severe |
| 112. | CALCIUM HYDROXIDE | Severe |
| 113. | CALCIUM PYROPHOSPHATE | Severe |
| 114. | CITRIC ACID | Severe |
| 115. | EDETIC ACID | Severe |
| 116. | ERYTHORBIC ACID | Severe |
| 117. | ETHYL ACETATE | Severe |
| 118. | FUMARIC ACID | Severe |
| 119. | GLUTAMIC ACID HYDROCHLORIDE | Severe |
| 120. | GLYCINE HYDROCHLORIDE | Severe |
| 121. | HYDROCHLORIC ACID | Severe |
| 122. | HYDROXYETHYL CELLULOSE | Severe |
| 123. | LACTIC ACID | Severe |
| 124. | MAGNESIUM OXIDE | Severe |
| 125. | MALEIC ACID | Severe |
| 126. | MALIC ACID | Severe |
| 127. | MYRISTIC ACID | Severe |
| 128. | OLEIC ACID | Severe |
| 129. | PALMITIC ACID | Severe |
| 130. | PHOSPHORIC ACID | Severe |
| 131. | PIPERAZINE | Severe |
| 132. | SODIUM BISULFATE | Severe |
| 133. | SULFURIC ACID | Severe |
| 134. | BUTYLATED HYDROXYANISOLE | Toxic |
| 135. | LEMON OIL | Toxic |
| 136. | LIME OIL | Toxic |
| 137. | LIMONENE, DL | Toxic |
| 138. | METHYL ALCOHOL | Toxic |
| 139. | NAPHTHA | Toxic |
| 140. | ORANGE PEEL EXTRACT | Toxic |
| 141. | TETRACHLOROETHYLENE | Toxic |

In one embodiment, the irritant or irritants are sufficient to induce moderate to severe coughing if a crushed dosage form of the present invention is inhaled. Specifically, as described above about 40% to about 70% of a crushed dosage form of the present invention passes completely through the nasal passages when inhaled. A portion of the crushed dosage form that is inhaled can then enter the lungs, and accordingly the one or more irritants included in a dosage form of the present invention can induce prolonged coughing after inhalation abuse.

Additionally, certain references indicate that dosage forms, and in particular dosage forms which may be administered by contact with nasal mucosa, should have particular pH, as described in Drug Delivery Technologies, Development of Nasal Delivery Systems: A Review, Jack Aurora, PhD, which can be found at www.drugdeliverytech.com, and in particular http://www.drugdeliverytech.com/cgi-bin/articles.cgi?idArticle=85, the content of which is hereby incorporated by reference.

In one embodiment, the one or more irritants (and/or other abuse deterrents) and excipients for use in the present invention combine to form a product of the present invention having an acidic (e.g., <about 7.0) pH. In one embodiment, the pH of embodiments of the invention can be less than 4, normally between 0 and 4, more typically between about 3 to 4. The reduced pH has an effect similar to a hypertonic solution on the tissues of the body. Additionally, the lower pH can cause shrinkage of the epithelial cells and thereby decrease drug absorption. The acidic pH of an embodiment of the present invention can also cause irritation as well as swelling of the nasal mucosa if a crushed dosage form of the present invention is inhaled.

In certain embodiments, additional pharmaceutically acceptable acidic excipient can also be used to lower the pH of dosage forms of the present invention. Suitable excipients include citric acid.

A comparison of the pH of an embodiment of the present invention described in Example 44 with commercially available products is provided in the following table:

pH Comparison Table

| Product | pH |
|---|---|
| Mallinckrodt 5 mg Oxycodone | 5.86 |
| Hydrochloride Tablet | |
| Purdue Pharmaceuticals 40 mg | 6.42 |
| OxyContin | |
| 5 mg Oxycodone Hydrochloride | 3.83 |
| Present Invention Tablet | |

D. Emetics

As described above, the present invention can include one or more emetics or emesis inducing agents. Preferably, the emetic is a pharmaceutically acceptable agent that only induces emesis after a certain threshold amount is ingested. In another embodiment, the emetic can be a pharmaceutically active emetic.

In one embodiment, the amount of emetic present in a pharmaceutical composition of the present invention can be tied directly to the amount of drug in the pharmaceutical composition. Thus, by controlling the quantity of the emetic compound in the pharmaceutical composition, emesis can be avoided if normal prescription directions are followed. However, if an overdosage occurs by ingesting more than a prescribed quantity of a drug in a pharmaceutical composition of the present invention, the amount of ingested emetic will exceed the threshold amount necessary to induce emesis.

In some embodiments, the threshold amount of emetic for inducing emesis can be reached when the normal prescription dosages are (e.g., a unit dosage) increased by factors of 2, 3, 4, 5, 6, 7, or 8 times, or more. Thus, in some embodiments, the amount of emetic present in a pharmaceutical composition of the present invention is an amount such that the amount of emetic ingested does not exceed the threshold amount necessary for inducing emesis until a subject ingests 2, 3, 4, 5, 6, 7, or 8 or more times the amount of drug normally prescribed. In some embodiments, emesis can preclude death or serious illness in the subject.

In one embodiment, the emetic includes zinc sulfate. Zinc sulfate is commonly referred to as an excipient, but can induce emesis when more than about 0.6 to 2.0 gm is ingested, typically more than about 0.6 gm. In one embodiment, a pharmaceutically acceptable agent which can induce emesis (e.g., zinc sulfate) can be present at about 5 to 60 percent by weight on a solid basis, or about 5 to 40 percent by weight on a solid basis or about 5 to 25 percent by weight on a solid basis more typically about 5 to 10 percent by weight on a solid basis.

Accordingly, pharmaceutical compositions of the present invention can be easily designed to induce emesis if a prescribed dosage is exceeded and/or if prescription directions are not followed for dosage forms containing a composition of the present invention. In some embodiments of the present invention, a dosage form can include about 0.01, 0.05, 0.1, 0.15, 0.2, 0.25, 0.3, 0.35, 0.4, 0.45, 0.5, 0.55, 0.6, 0.65, 0.7, 0.75, 0.8, 0.85, 0.90, 0.95, 1.0 grams of a pharmaceutically acceptable agent which can induce emesis (e.g., zinc sulfate) or pharmaceutically active emetic. In another embodiment, the present invention includes an agent which can induce emesis (e.g., zinc sulfate) and/or a pharmaceutically active emetic in an amount that is a summation of two or more of the above described amounts.

In another embodiment, the present invention can include 1, 2, 3, 4, or 5 times, or more, of the above described amounts of pharmaceutically acceptable agent which can induce emesis (e.g., zinc sulfate) and/or a pharmaceutically active emetic. Typically, suitable embodiments of the present invention include from about 0.1 gm to about 2.0 gm of zinc sulfate per amount of drug normally prescribed (e.g., unit dosage). In other embodiments the present invention can include about 0.6 to less than about 2.0 gm of zinc sulfate per amount of drug normally prescribed.

For example, in one embodiment, if a practitioner desires to create a dosage form that will induce emesis only after four or more unit dosage forms are ingested, the amount of zinc sulfate in each dosage form should not exceed about 0.19 gm. Thus, if three dosage forms are ingested, the amount of emetic is 0.57 gm, which is less than a typical threshold amount of the particular emetic. However, if a fourth dosage form having 0.19 gm. of zinc sulfate is ingested, the amount of emetic exceeds the threshold amount, and emesis is induced.

The above-described emetics can be further optimized as necessary or desired in terms of concentration in the pharmaceutical composition, etc.

Other emetics which can be suitable for use in the present invention which can be administered in sub-therapeutic amounts include one or more of cephaeline, methyl cephaeline, psychotrine, O-methylpsychotrine, ammonium chloride, potassium chloride, magnesium sulfate, ferrous gluconate, ferrous sulfate, aloin, and emetine.

E. Laxative/Stool Softener

In one embodiment, the invention includes a laxative/stool softener in a dosage form of the present invention. In one embodiment, the present invention includes an amount of a laxative/stool softener such that the laxation/stool softening effect does not occur until more than a prescribed dosage (e.g., a unit dosage) of the pharmaceutical agent susceptible to abuse (e.g., an analgesic) is consumed.

Accordingly, in one embodiment the amount of laxative/stool softener present in a pharmaceutical composition of the present invention can be tied directly to the amount of drug in the pharmaceutical composition. Thus, by controlling the quantity of the laxative/stool softener compound in the pharmaceutical composition, laxation can be avoided if normal prescription directions are followed. However, if an overdosage occurs by ingesting more than a prescribed quantity of a drug in a pharmaceutical composition of the present invention, the total amount of ingested laxative/stool softener will, in certain embodiments, exceed the threshold amount necessary to induce laxation/stool softening.

Accordingly, the amount of laxative/stool softener in a dosage form of the present invention can vary depending upon the choice of laxative/stool softener. Typically, the amount of laxative/stool softener included in a dosage form of the present invention is less than an effective amount of the laxative/stool softener (i.e., less than the threshold amount).

In certain embodiments, a dosage form of the present invention includes an anionic surfactant as a laxative/stool softener. In one embodiment, the anionic surfactant includes sodium dioctyl sulfosuccinate (docusate), as described in U.S. patent application Ser. No. 10/716,163 to Mayo-Alvarez et al., U.S. Application Publication No. 2004/0151791, the entire content of which is hereby incorporated by reference. In one embodiment, the present invention can include about 10 mg to 300 mg of sodium dioctyl sulfosuccinate. In another embodiment, the dosage form of the present invention includes about 25 mg to 200 mg, or between 50 mg to about 100 mg, of sodium dioctyl sulfosuccinate. In further embodiments of the present invention, a dosage form of the present invention includes calcium and/or potassium dioctyl sulfosuccinate.

In another embodiment, senna/sennosides (the active ingredient in ExLax®), magnesium citrate, magnesium sulfate, olestra, aloin (aloe component), dehydrocholic acid, cascara, and plantago seed can be used. Other suitable ingredients that can be used in a dosage form of the present invention in the manner described above include magnesium hydroxide, polyethylene glycol 400, mannitol, and sorbitol. The threshold amount of the above described ingredients suitable for causing laxation/stool softening is apparent to one skilled in the art. Accordingly, in preferred embodiments of the invention, it is desirable to include less than a threshold amount of a laxative/stool softening agent (i.e., a sub-therapeutic amount).

F. Tissue Staining Agents

In another embodiment, the present invention includes one or more tissue staining agents including dyes such as tissue staining dyes. In one embodiment, the staining agent can be water soluble (dyes) or oil soluble (e.g., water insoluble or “lake”). In preferred embodiments, the staining agent can be water soluble.

A staining agent can be included in a dosage form of the present invention in order to prevent, reduce or inhibit abuse of the active pharmaceutical ingredient of the dosage form. In one embodiment, the staining agent is mixed with the active pharmaceutical ingredient and other constituents of the present invention. In another embodiment the staining agent can be sequestered from the other constituents of the dosage form of the present invention, as described further below. With respect to certain embodiments, it should also be noted that the tissue staining agent can be encapsulated or sequestered in a film coating or polymer using techniques apparent to one of skill in the art, such that when used in a prescribed manner, the tissue staining agent will not be exposed to external and/or visible stainable tissue.

The staining agent prevents, reduces or inhibits abuse of the active pharmaceutical ingredient by staining the tissues that come into contact with the staining agent. Typically, a staining agent is included in a dosage form of the present invention and apparent staining does not occur during normal use of the dosage form. However, after a dosage form is tampered with (e.g., by crushing), the staining agent is exposed and can stain tissues that contact the tissue staining agent. For example in one embodiment, the fingers of an abuser can be stained upon touching a crushed dosage form of the present invention. In another embodiment, the nose and/or area in or about the nose of an abuser can be stained upon nasal inhalation of a crushed dosage form of the present invention.

In certain embodiments of the present invention, suitable tissue staining agents can include one or more of the following: FD&C Blue No. 1 (Dye and Lake), FD&C Blue No. 2 (Dye and Lake), FD&C Green No. 3 (Dye and Lake), FD&C Red No. 3 (Dye), FD&C Red No. 40 (Dye and Lake), FD&C Yellow No. 5 (Dye and Lake), FD&C Yellow No. 6 (Dye and Lake), Orange B, Citrus Red No. 2, carbon black, annatto extract, beta-carotene, canthaxanthin, carrot oil, cottonseed flour (toasted partially defatted, cooked), ferrous gluconate, grape color extract, turmeric oleoresin, B-Apo-8′-carotenal, beet powder, caramel color, cochineal extract (carmine), fruit juice, grape skin extract (enocianina), paprika oleoresin, saffron, turmeric, vegetable juice, acid fuchsin, acridine orange, alcian blue 8gx, alizarin red s, aniline blue, auramine o, azocarmine g, azur a, azur b azur ii, basic fuchsin (rosaniline), basic green 4 (malachite green), biebrich scarlet (ponceau bs), bismarck brown y, brilliant cresyl blue, carmine (alum lake), cresyl fast violet, (cresyl violet acetate), crystal violet, eosin y, erythrosin b (erythrosin extra bluish), fast green fcf, fluorescein isothiocyanate, giemsa (dry powder), hematoxylin, indigo carmine, light green sf, yellowish, methyl green, methylene blue, methyl violet 2b, nigrosin, w.s., nile blue a, orange ii, orange g, phloxine b, phloxine b, safranin o, sudan black b, toluidine blue o, and wright stain.

In certain preferred embodiments, the present invention can include the dyes set forth in U.S. Patent Application Publication No. 20040228802, to Chang et al., the entire content of which is hereby incorporated by reference. Such dyes include allura red, amaranth, brilliant blue, canthaxanthin, carmine, carmoisine, carotene, curcumin, erythrosine, green S, indigo carmine, iron oxide black, iron oxide red, iron oxide yellow, patent blue, phloxine O, ponceau 4R, quinoline yellow, riboflavin, sunset yellow, tartrazine, titanium dioxide, vegetable carbon black, and other natural colors such as annatto, beet, black carrot, black currant, caramel, carmine, carmine lake, chlorophyll, cochineal, elderberry, grapeskin/grape juice, malt, paprika, red cabbage, turmeric, and anthocyanins. In certain embodiments, riboflavin is a preferred indicator because it can also be used as a tracing agent for easy urine detection of drug abusers.

The amount of the dye used in a dosage form of the present invention will vary with the particular dye used but, typically, the dye indicator is used in an amount of 0.01 to 20% by weight and, preferably, 0.1 to 10% by weight, and, most preferably, 0.1 to 5% by weight, based on the weight of a dosage form.

G. Malodorous/Repugnant Agents

In one embodiment, the present invention includes a substance which is malodorous, repugnant or pungent to the sense of smell.

In one embodiment, suitable organic compounds contain the group —SH bonded to a carbon atom. In one embodiment, volatile low-molecular-weight mercaptans can be used. Several suitable mercaptans and thiols are listed in the GRAS/EAFUS database. Other suitable constituents can include butyric acid, 3-Methylbutanoic acid (isovaleric acid) hydrogen sulfide, ammonia, cadaverine, and putricene, as well as menhaden oil, and cod liver oil.

Corresponding pungent agents are known to the person skilled in the art and are described, for example, in Pharmazeutische Biologie—Drogen and ihre Inhaltsstoffe [Pharmaceutical Biology—Drugs and their Constituents], 2nd, revised edition, Gustav Fischer Verlag, Stuttgart-N.Y., 1982, the entire content of which is hereby incorporated by reference.

In one embodiment, a dosage form according to the present invention may preferably contain a pungent agent in an amount of 0.01 wt. % to 30 wt. % and especially preferably 0.1 wt. % to 0.5 wt. %, always relative to the total weight of the dosage unit.

If one or more constituents of corresponding pungent agents are used, their amount in the dosage form according to the present invention is preferably between 0.001 wt. % and 0.005 wt. % relative to the total weight of the dosage unit.

In one embodiment, a dosage form according to the present invention includes one or more constituents of at least one pungent agent, selected from the group of allii sativi bulbus, asari rhizoma c. herba, calami rhizoma, capsici fructus (paprika), capsici fructus acer (cayenne pepper), curcumae longae rhizoma, curcumae xanthorrhizae rhizoma, galangae rhizoma, myristicae semen, piperis nigri fructus (pepper), sinapis albae (erucae) semen, sinapis nigri semen, zedoariae rhizoma and zingiberis rhizoma, especially preferably from the group comprising capsici fructus (paprika), capsici fructus acer (cayenne pepper) and piperis nigri fructus (pepper).

In another embodiment, the constituents of the pungent agent are o-methoxy(methyl) phenol compounds, mustard oils or sulfide derivatives or compounds derived therefrom.

In yet another embodiment, a constituent of the pungent agent is selected from the group of myristicin, elemicin, isoeugenol, beta-asarone, saffrole, gingerols, xanthorrhizol, capsaicinoids, preferably capsaicin, piperine, preferably trans-piperine, glucosinolates. In another embodiment, pungent agents include agents based on nonvolatile mustard oils, preferably those based on p-hydroxybenzyl mustard oil, methyl mercapto mustard oil or methyl sulfonyl mustard oil, and derivatives thereof.

In one embodiment, the pungent agent is sequestered such that unless the dosage form is tampered with (e.g., crushed) the pungent agent is not released, as described further below. Preferably, unless tampered with, the sequestered pungent agent passes through the body without being released (i.e. the pungent agent remains sequestered).

H. Flushing, Discomfort and/or Pain Inducing Agents

In one embodiment, the present invention includes an agent that induces flushing, (i.e. redness of the skin, including redness of the skin of one or more of the face, neck, chest, back and trunk and legs) and/or itching and/or discomfort and/or temporary pain (a flushing/pain/headache inducing agent or flushing/headache inducing agent), and/or generalized pruritis, and/or intense warmth, and/or chills when administered at or in excess of a threshold amount. In one embodiment, the pain is a headache.

As described above, with respect to flushing, discomfort and pain inducing agents, in the present invention, a threshold amount is an amount below which one or more adverse effects is absent or below which a subject may experience a beneficial effect.

In one embodiment, the flushing agent and/or itching agent and/or pain inducing agent is a drug. In certain embodiments, the drug is obtainable “over the counter” and in certain embodiments, the “over the counter” drug is a vitamin. In yet another embodiment, the vitamin is niacin, which can be commercially purchased under the tradenames “Niaspan®” and “Niacor®”. In another embodiment, the present invention includes vitamin A.

Accordingly, in one embodiment the amount of flushing/itching/headache inducing agent present in a pharmaceutical composition of the present invention can be tied directly to the amount of drug in the pharmaceutical composition. Thus, by controlling the quantity of the flushing agent and/or itching agent and/or pain inducing agent in the pharmaceutical composition, flushing and/or headache can be avoided if normal prescription directions are followed. However, if an overdosage occurs by ingesting more than a prescribed quantity of a drug in a pharmaceutical composition of the present invention (e.g., by ingesting more than the prescribed dose), the total amount of flushing/headache inducing agent can, in certain embodiments, exceed the threshold amount necessary to induce flushing and/or itching and/or headache thereby inducing flushing and/or itching and/or headache.

In one embodiment, the present invention includes about 10 mg to about 500 mg of the flushing/headache inducing agent. In yet another embodiment, the present invention includes about 15 mg to about 150 mg of flushing/pain/headache the present invention includes about 50 mg to about 150 mg of the flushing/pain/headache inducing agent. In another embodiment, the present invention includes 15, 30, 45, 60, 75, 90 or 105 mg of the flushing/pain/headache inducing agent. In one embodiment, the present invention includes a flushing/pain/headache inducing agent in an amount of about 1% to 25%, typically about 3% to 15%, more typically about 1%, 3%, 6%, 9%, 12%, 15% or 20% by weight, including or excluding the weight of any analgesic and/or other drug susceptible to abuse. Examples 35 to 42 provide placebo (i.e., free of analgesic) embodiments of the present invention.

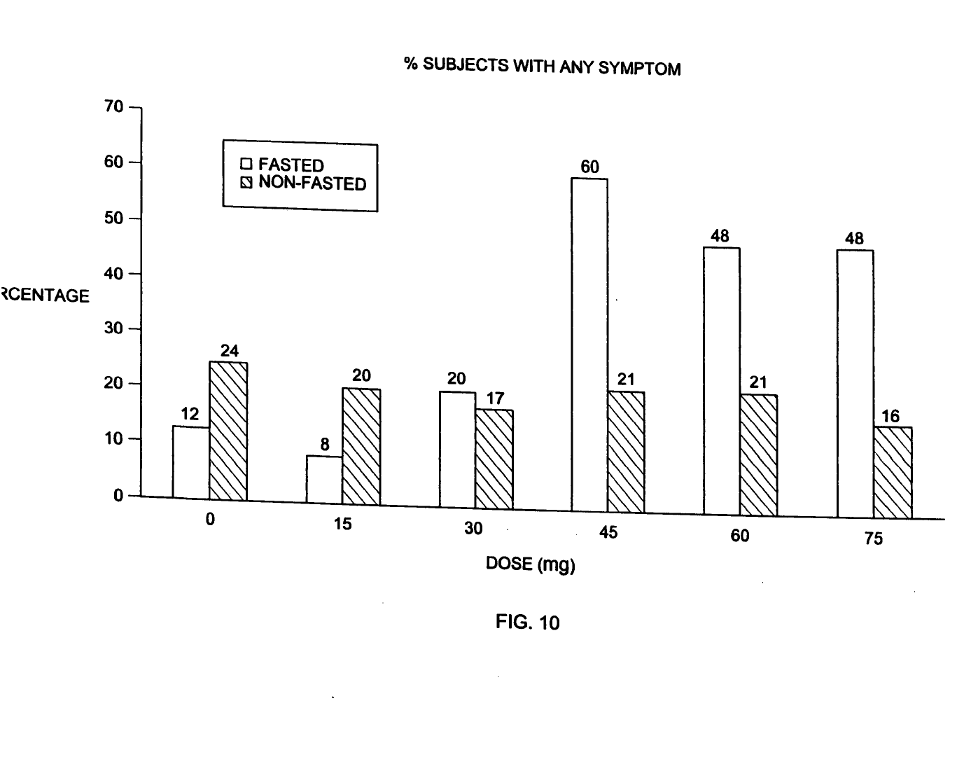

In some embodiments, as shown in FIG. 10, the amount of flushing/pain/headache inducing agent can be from about 15 to about 75 mg. As shown in FIG. 10, in a fasted state and at an administered dose of about 45 mg of a flushing/pain/headache inducing agent, a substantial number of subjects indicated aversive symptoms. This 45 mg value corresponds to about the threshold level of certain flushing/pain/headache inducing agents, and the value corresponds to a therapeutic dose of certain flushing/pain/headache inducing agents.

Accordingly, in one embodiment of the present invention, about 30 mg of a flushing/pain/headache inducing agent can be administered with a prescribed dose of a drug without inducing substantial aversive symptoms and accordingly corresponds to a sub-therapeutic dose of certain flushing/pain/headache inducing agents. However, if the consumed dose of the drug meets or exceeds the prescribed dose, aversive symptoms are induced.

In another embodiment, the flushing agent and/or itching agent and/or pain inducing agent can be an FDA approved active pharmaceutical (other than the drug or drugs in the dosage form that are susceptible to abuse, e.g., oxycodone) which itself requires a prescription or that is highly pharmaceutically active that induces flushing, itching, and/or pain or discomfort when a threshold amount is reached or exceeded during administration. Accordingly, in preferred embodiments, a dosage form of the present invention includes a sub-therapeutic amount of a flushing agent and/or itching agent and/or pain or discomfort inducing agent. In another embodiment, the amount of drug present in a dosage form should be an amount sufficient to cause one or more of flushing, pain or discomfort or itching if the dosage form is abused (e.g., an overdosage occurs) or if a threshold amount of the agent is reached or exceeded during administration.

In one embodiment the active pharmaceutical includes atropine sulfate. In an embodiment where the flushing/pain/headache inducing agent is atropine sulfate, the amount of atropine sulfate in a single dosage form of the present invention can typically be about 0.02 mg to 1.0 mg.

It should be noted that in certain embodiments, and in particular dosage forms having controlled release, the amount of flushing agent (and in other embodiments, the amount of any abuse deterrent component described herein), can exceed the threshold amount present in an immediate release form. This is because in controlled release formulations, the amount of drug which is susceptible to abuse is typically higher than in an immediate release formulation and the flushing agent (or other abuse deterrent component) becomes bioavailable at a slower rate than the immediate release form. Thus, the amount of abuse deterrent component which is bioavailble typically also remains below the amount sufficient to cause an abuse deterrent effect. However, if the dosage form is tampered with (e.g., ground, chewed or crushed), a large portion of the abuse deterrent component becomes immediately bioavailable, thus inducing one or more abuse deterrent effects.

I. Other Ingredients

The present invention can also optionally include other ingredients to enhance dosage form manufacture from a pharmaceutical composition of the present invention and/or alter the release profile of a dosage foaming including a pharmaceutical composition of the present invention.

Some embodiments of the present invention include one or more pharmaceutically acceptable fillers/diluents. In one embodiment, Avicel PH (Microcrystalline cellulose) is a filler used in the formulation. The Avicel PH can have an average particle size ranging from 20 to about 200 μm, preferably about 100 μm. The density ranges from 1.512-1.668 g/cm³. The Avicel PH should have molecular weight of about 36,000. Avicel PH effectiveness is optimal when it is present in an amount of from about 10 to 65 percent, by weight on a solid basis, of the formulation. Typical fillers can be present in amounts from 10 to 65 percent by weight on a dry weight basis of the total composition. Other ingredients can include sugars and/or polyols. In certain embodiments, the present invention includes about 355, 340, 325, 310, 295 or 280 mg. of Avicel.

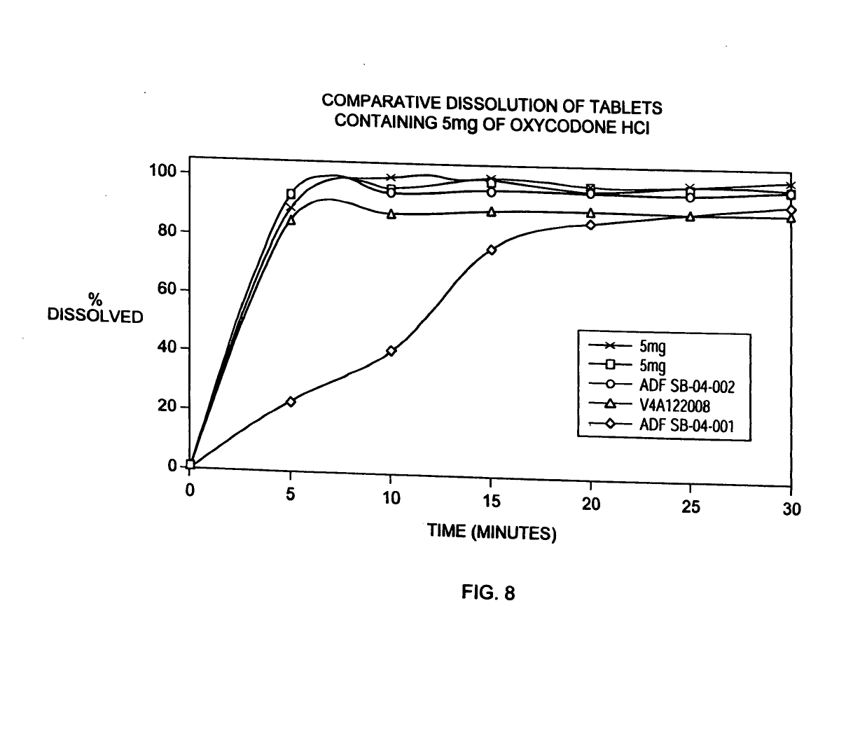

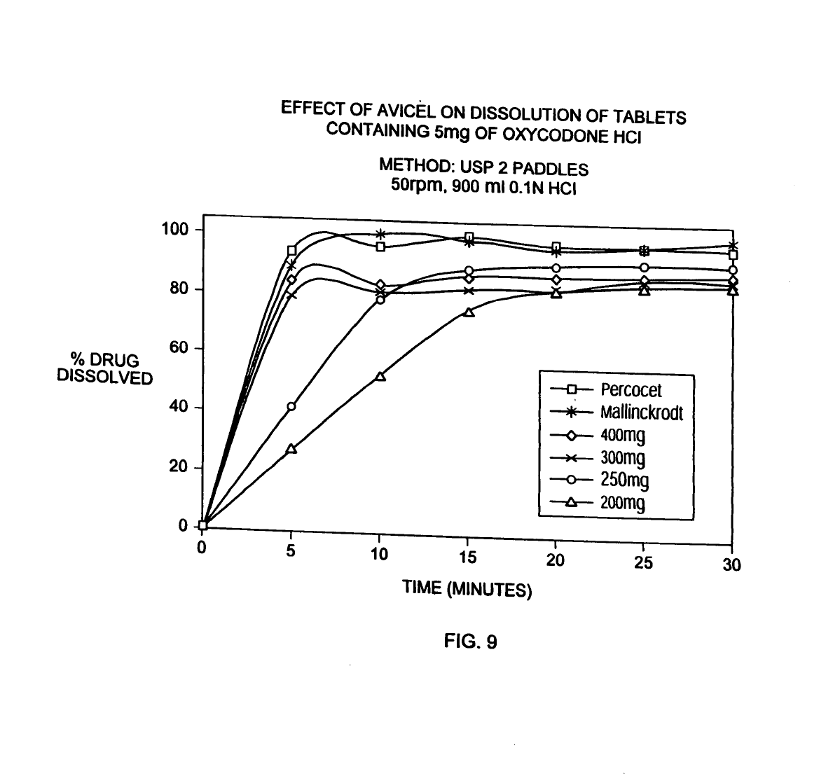

As shown in FIGS. 8 and 9, in the present invention the amount of Avicel included in certain embodiments can have an effect on dissolution. With respect to FIGS. 8 and 9, it should be noted that the Percocet and Mallinckrodt lines are provided for reference purposes only and represent commercially available products.

In FIG. 8, ADF SB-04-001 included 150 mg of zinc sulfate and 200 mg of Avicel, prepared in accordance with Example 29. As also shown in FIG. 8, V4A122008 included 100 mg zinc sulfate and 250 mg of Avicel, prepared in accordance with Example 28. Additionally, ADF SB-04-002 included 50 mg of zinc sulfate and 300 mg of Avicel, prepared in accordance with Example 6. Accordingly, as shown in FIG. 8, as the amount of Avicel increased, the dissolution of the tablet also increased.

Further, as shown by FIG. 9, where zinc sulfate was held constant at 150 mg in the non-commercially available tablets, it is believed that the Avicel, and not the zinc sulfate controls the rate of tablet dissolution. Specifically, as shown in FIG. 9, as the amount of Avicel was increased from 200 mg to 250 mg to 300 mg, to 400 mg, the rate of dissolution also increased.

Other ingredients can also include dibasic calcium phosphate having a particle size of about 75 to about 425 microns and a density of about 0.5 to about 1.5 g/ml, as well as calcium sulfate having a particle size of about 1 to about 200 microns and a density of about 0.6 to about 1.3 g/ml and mixtures thereof. Further, lactose having a particle size of about 20 to about 400 microns and a density of about 0.3 to about 0.9 g/ml can also be included.

In some embodiments of the invention, the fillers which can be present at about 10 to 65 percent by weight on a dry weight basis, also function as binders in that they not only impart cohesive properties to the material within the formulation, but can also increase the bulk weight of a directly compressible formulation (as described below) to achieve an acceptable formulation weight for direct compression. In some embodiments, additional fillers need not provide the same level of cohesive properties as the binders selected, but can be capable of contributing to formulation homogeneity and resist segregation from the formulation once blended. Further, preferred fillers do not have a detrimental effect on the flowability of the composition or dissolution profile of the formed tablets.

In one embodiment, the present invention can include one or more pharmaceutically acceptable disintegrants. Such disintegrants are known to a skilled artisan. In the present invention, disintegrants can include, but are not limited to, sodium starch glycolate (Explotab®) having a particle size of about 104 microns and a density of about 0.756 g/ml, starch (e.g., Starch 21) having a particle size of about 2 to about 32 microns and a density of about 0.462 g/ml, Crospovidone® having a particle size of about 400 microns and a density of about 1.22 g/ml, and croscarmellose sodium (Ac-Di-Sol) having a particle size of about 37 to about 73.7 microns and a density of about 0.529 g/ml. The disintegrant selected should contribute to the compressibility, flowability and homogeneity of the formulation. Further the disintegrant can minimize segregation and provide an immediate release profile to the formulation. In some embodiments, the disintegrant (s) are present in an amount from about 2 to about 25 percent by weight on a solid basis of the directly compressible formulation.

In one embodiment, the present invention can include one or more pharmaceutically acceptable glidants, including but not limited to colloidal silicon dioxide. In one embodiment, colloidal silicon dioxide (Cab-O—Sil®) having a density of about 0.029 to about 0.040 g/ml can be used to improve the flow characteristics of the formulation. Such glidants can be provided in an amount of from about 0.1 to about 1 percent by weight of the formulation on a solid basis. It will be understood, based on this invention, however, that while colloidal silicon dioxide is one particular glidant, other glidants having similar properties which are known or to be developed could be used provided they are compatible with other excipients and the active ingredient in the formulation and which do not significantly affect the flowability, homogeneity and compressibility of the formulation.

In one embodiment, the present invention can include one or more pharmaceutically acceptable lubricants, including but not limited to magnesium stearate. In one embodiment, the magnesium stearate has a particle size of about 450 to about 550 microns and a density of about 1.00 to about 1.80 g/ml. In one embodiment, magnesium stearate can contribute to reducing friction between a die wall and a pharmaceutical composition of the present invention during compression and can ease the ejection of the tablets, thereby facilitating processing. In some embodiments, the lubricant resists adhesion to punches and dies and/or aid in the flow of the powder in a hopper and/or into a die. In an embodiment of the present invention, magnesium stearate having a particle size of from about 5 to about 50 microns and a density of from about 0.1 to about 1.1 g/ml is used in a pharmaceutical composition. In certain embodiments, a lubricant should make up from about 0.1 to about 2 percent by weight of the formulation on a solids basis. Suitable lubricants are stable and do not polymerize within the formulation once combined. Other lubricants known in the art or to be developed which exhibit acceptable or comparable properties include stearic acid, hydrogenated oils, sodium stearyl fumarate, polyethylene glycols, and Lubritab®.

In certain embodiments, the most important criteria for selection of the excipients are that the excipients should achieve good content uniformity and release the active ingredient as desired. The excipients, by having excellent binding properties, and homogeneity, as well as good compressibility, cohesiveness and flowability in blended form, minimize segregation of powders in the hopper during direct compression.

In another embodiment, the present invention can include an opioid antagonist in addition to the other ingredients, or as a substitute for one of the other abuse deterrent ingredients of a formulation of the present invention. Suitable antagonists are described above. One particular antagonist includes naloxone. As described above, typically naloxone has no action when taken orally, and will not interfere with the pharmacologic action of an opioid agonist. However, when given by injection naloxone can have profound antagonistic action to opioid agonists. An appropriate antagonist can be used in combination with one or more of gel forming agents, mucous membrane irritants and/or nasal passageway tissue irritants, or emetics in the present invention. An appropriate antagonist can also be used as a substitute for one or more of gel forming agents, mucous membrane irritants and/or nasal passageway tissue irritants, or emetics in the present invention. Suitable opioid receptor antagonists can include but are not limited to the antagonists described in U.S. Pat. Nos. 6,559,159 and 6,375,957, the entire content of which are hereby incorporated by reference. Further, in preferred embodiments, the antagonist is sequestered such that the antagonist is not released unless the dosage form is tampered with, such as by crushing. Techniques suitable for sequestering one or more components (which can include a drug and/or one or more deterrents, described above) in a dosage form of the present invention are believed to be apparent to a skilled artisan.

J. Sequestered Subunits

In certain embodiments, one or more of the above described components of the present invention, including a drug or abuse deterrent agent such as gel forming agents, mucous membrane irritants, emetics, stool softeners, tissue staining agents, malodorous/repugnant agents, flushing agents and pain or discomfort agents, may be sequestered in the manner as described in U.S. Patent Publication No. 20030125347, to Anderson et al., the entire content of which is hereby incorporated by reference. The term “sequestered” is defined for purposes of the present invention as physically isolated and/or chemically bound and biologically unavailable. If, however, the integrity of the dosage form is destroyed such as by physical destruction or dissolution, which is another mode of use associated with opiate abuse, then the sequestered component can be released from sequestration. In certain embodiments, the component is sequestered by using a material that is a polymer that is insoluble in the gastrointestinal tract.

Suitable polymers for sequestration of one or more components of the present invention are set forth in U.S. Patent Application Publication No. 20040131552, to Boehm, the entire content of which is hereby incorporated by reference, and include a cellulose or an acrylic polymer. Desirably, the cellulose is selected from the group consisting of ethylcellulose, cellulose acetate, cellulose propionate, cellulose acetate propionate, cellulose acetate butyrate, cellulose acetate phthalate, cellulose triacetate, and combinations thereof. Ethylcellulose includes, for example, one that has an ethoxy content of about 44 to about 55%. Ethylcellulose can be used in the form of an aqueous dispersion, an alcoholic solution, or a solution in other suitable solvents. The cellulose can have a degree of substitution (D.S.) on the anhydroglucose unit, from greater than zero and up to 3 inclusive. By “degree of substitution” is meant the average number of hydroxyl groups on the anhydroglucose unit of the cellulose polymer that are replaced by a substituting group. Representative materials include a polymer selected from the group consisting of cellulose acylate, cellulose diacylate, cellulose triacylate, cellulose acetate, cellulose diacetate, cellulose triacetate, monocellulose alkanylate, dicellulose alkanylate, tricellulose alkanylate, monocellulose alkenylates, dicellulose alkenylates, tricellulose alkenylates, monocellulose aroylates, dicellulose aroylates, and tricellulose aroylates.

More specific celluloses include cellulose propionate having a D.S. of 1.8 and a propyl content of 39.2 to 45 and a hydroxy content of 2.8 to 5.4%; cellulose acetate butyrate having a D.S. of 1.8, an acetyl content of 13 to 15% and a butyryl content of 34 to 39%; cellulose acetate butyrate having an acetyl content of 2 to 29%, a butyryl content of 17 to 53% and a hydroxy content of 0.5 to 4.7%; cellulose triacylate having a D.S. of 2.9 to 3, such as cellulose triacetate, cellulose trivalerate, cellulose trilaurate, cellulose tripatmitate, cellulose trisuccinate, and cellulose trioctanoate; cellulose diacylates having a D.S. of 2.2 to 2.6, such as cellulose disuccinate, cellulose dipalmitate, cellulose dioctanoate, cellulose dipentanoate, and coesters of cellulose, such as cellulose acetate butyrate, cellulose acetate octanoate butyrate, and cellulose acetate propionate.

Additional cellulose polymers useful for the invention include acetaldehyde dimethyl cellulose acetate, cellulose acetate ethylcarbamate, cellulose acetate methycarbamate, and cellulose acetate dimethylaminocellulose acetate.

The acrylic polymer preferably is selected from the group consisting of methacrylic polymers, acrylic acid and methacrylic acid copolymers, methyl methacrylate copolymers, ethoxyethyl methacrylates, cyanoethyl methacrylate, poly(acrylic acid), poly(methacrylic acid), methacrylic acid alkylamide copolymer, poly(methyl methacrylate), polymethacrylate, poly(methyl methacrylate) copolymer, polyacrylamide, aminoalkyl methacrylate copolymer, poly(methacrylic acid anhydride), glycidyl methacrylate copolymers, and combinations thereof. An acrylic polymer useful for preparation of a sequestering subunit of the invention includes acrylic resins comprising copolymers synthesized from acrylic and methacrylic acid esters (e.g., the copolymer of acrylic acid lower alkyl ester and methacrylic acid lower alkyl ester) containing about 0.02 to about 0.03 mole of a tri (lower alkyl) ammonium group per mole of the acrylic and methacrylic monomer used. An example of a suitable acrylic resin is ammonio methacrylate copolymer NF21, a polymer manufactured by Rohm Pharma GmbH, Darmstadt, Germany, and sold under the Eudragit® trademark. Eudragit RS30D is preferred. Eudragit® is a water-insoluble copolymer of ethyl acrylate (EA), methyl methacrylate (MM) and trimethylammoniumethyl methacrylate chloride (TAM) in which the molar ratio of TAM to the remaining components (EA and MM) is 1:40. Acrylic resins, such as Eudragit® can be used in the form of an aqueous dispersion or as a solution in suitable solvents.

In another preferred embodiment, the sequestering material is selected from the group consisting of polylactic acid, polyglycolic acid, a co-polymer of polylactic acid and polyglycolic acid, and combinations thereof. In certain other embodiments, the hydrophobic material includes a biodegradable polymer comprising a poly(lactic/glycolic acid) (“PLGA”), a polylactide, a polyglycolide, a polyanhydride, a polyorthoester, polycaprolactones, polyphosphazenes, polysaccharides, proteinaceous polymers, polyesters, polydioxanone, polygluconate, polylactic-acid-polyethylene oxide copolymers, poly(hydroxybutyrate), polyphosphoester or combinations thereof.

Preferably, the biodegradable polymer comprises a polylactic/glycolic acid), a copolymer of lactic and glycolic acid, having a molecular weight of about 2,000 to about 500,000 daltons. The ratio of lactic acid to glycolic acid is preferably from about 100:1 to about 25:75, with the ratio of lactic acid to glycolic acid of about 65:35 being more preferred.

The component may be sequestered in a variety of ways all of which are considered within the scope of the invention. Physical sequestration may be achieved, for example, by coating the component in a pharmaceutically acceptable material that forms a substantially indigestible barrier. The coated component is then combined with the opiate to form an embodiment of a dosage form of the present invention. Sequestration may be accomplished also by the formation of chemical bonds between the component and a pharmaceutically acceptable material, such as for example a chelating agent, such that the component is rendered biologically unavailable to the patient when taken as directed as a part of a dosage form. Whether physical and/or chemical sequestration is employed, the manner of sequestration is selected so that the component is released from sequestration if the physical barrier or the chemical bonds of the sequestering agent is compromised. As noted above, the release of sequestered component may be accomplished physically, for example, by crushing, or chemically, for example, by a solvent capable of degrading the sequestering material or breaking the bonds with the component. By the selection of sequestering agents which are capable of releasing a particular component by means of the same methods that are associated with abuse of pharmaceutical forms of opiates, the sequestration of one or more deterrents (e.g., a malodorous/repugnant agent and/or a tissue staining agent) is specifically designed to deter such abuse.

In certain embodiments, the dosage form of the present invention includes at least two populations of sequestered subunits. In other embodiments, the dosage form can include at least three populations of sequestered subunits. In certain embodiments, a sequestered subunit including one or more abuse deterrent agents is indistinguishable from a sequestered subunit of another population (e.g., a subunit including a drug susceptible to abuse). In certain embodiments, a sequestered subunit including one or more abuse deterrent agents is physically indistinguishable (e.g., in size and/or shape and/or color) from a sequestered subunit of another population. In certain embodiments, the sequestered subunits are homogenous in shape and/or coloration and/or size and/or texture. In certain embodiments, the sequestered subunits include beads, pellets, granulates, particles, or microcapsules within the dosage form of the present invention. These embodiments deter ready separation of the dosage form components and selective extraction of a significant amount or even an entire amount of the drug for absorption by injection, inhalation, and/or oral consumption.

The construction of a dosage form including homogenous sequestered subunits of the present invention may occur by any means familiar to one of ordinary skill in the art. In certain embodiments, a sequestered subunit including one or more abuse deterrent agents may be specifically designed to be indistinguishable in size, shape, and color from a pre-existing subunit that includes a drug susceptible to abuse; and subsequently combined into a single dosage form. In certain embodiments, a subunit including a drug susceptible to abuse may be manufactured to be appear identical to, and subsequently combined with, a sequestered subunit including one or more abuse deterrents. In certain embodiments, both a sequestered subunit including one or more abuse deterrents and a sequestered subunit including a drug susceptible to abuse may be concurrently designed and manufactured to be indistinguishable in size, shape, and color; and subsequently combined into a single dosage form.

In certain embodiments, the present invention includes a homogenous mixture of two immediate release sequestered subunits including a first immediate release sequestered subunit including one or more abuse deterrents; and a second immediate release sequestered subunit including a drug susceptible to abuse. In certain embodiments, the dosage form of the present invention further includes a third extended release subunit, wherein the extended release subunit releases a drug susceptible to abuse and is indistinguishable in size and/or shape and/or color and/or texture from the first and/or second immediate release subunits.

K. Dosage Forms of the Present Invention

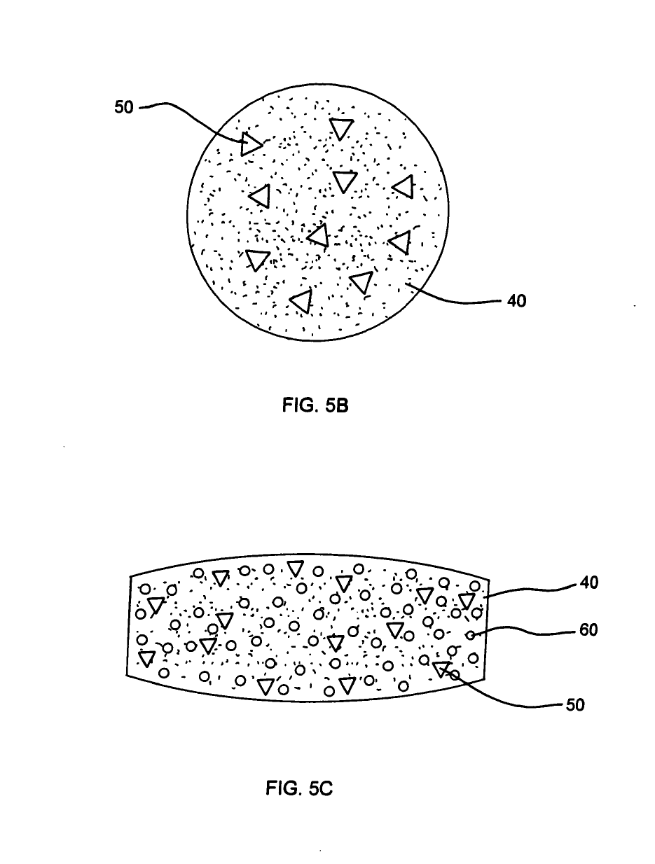

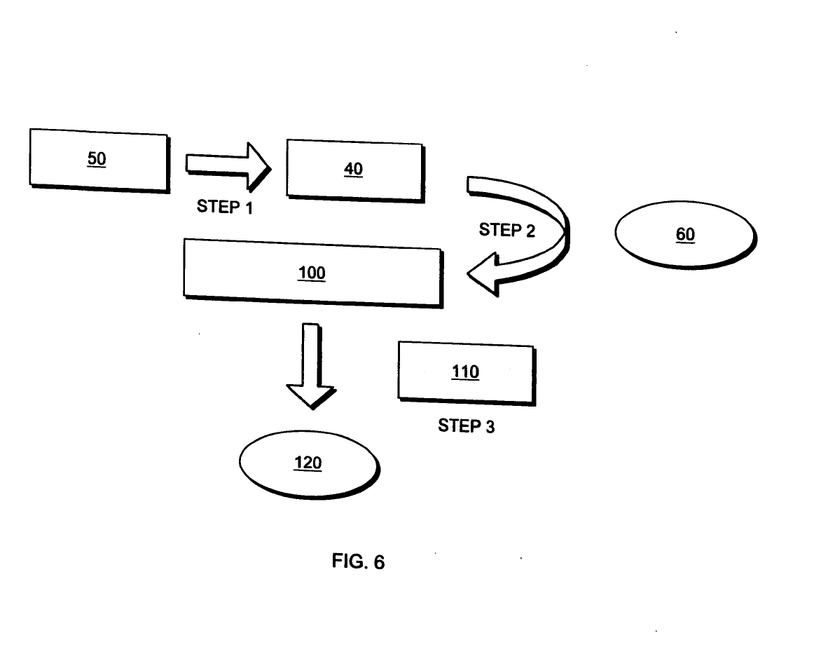

A pharmaceutical composition of the present invention including one or more drug components, one or more of gel forming agents, mucous membrane irritants and/or nasal passageway tissue irritants, and emetics, and optionally other ingredients, can be suitably modified and processed to form a dosage form of the present invention. As referred to herein and in FIGS. 5a, 5b, 5c and 6, an “abuse deterrent composition” or “ADC” (labeled “40” in these Figures) includes a composition having one or more gel forming agents and/or mucous membrane irritants and/or nasal passageway tissue irritants, and/or emetics according to the teachings set forth herein. In this manner, an abuse deterrent composition can be layered onto, coated onto, applied to, admixed with, formed into a matrix with, and/or blended with a drug and optionally other ingredients, thereby providing a therapeutic composition of the present invention.

As shown in FIG. 5a, an abuse deterrent composition can be combined with a drug and/or opioid analgesic (e.g., hydrocodone) in one or more layered dosage forms. According to the present invention, drug 50 can be a layer on or near the surface (I) of ADC 40 of the present invention, or sandwiched between two or more distinct layers (II and III) of ADC 40 of the present invention. In other embodiments, drug 50 can be a coating (IV) on ADC 40. Drug 50 can be any of the pharmaceutically active ingredients (e.g., opioids) described herein and can be combined with other excipients, e.g. disintegrants including but not limited to sodium starch glycolate or Explotab®.

As shown in FIG. 5b an abuse deterrent composition 40 of the present invention can be combined with drug 50, e.g., hydrocodone, in a blended mixture. In such embodiments, drug 50 and ADC 40 can be evenly mixed.

As shown in FIG. 5c abuse deterrent composition 40 of the present invention can be combined with drug 50, e.g., hydrocodone, in a blended mixture with other ingredients 60, e.g., a disintegrant.

FIG. 6 shows one embodiment of the present invention for making a dosage form of the present invention. Specifically, a first step (step 1) of FIG. 4 shows drug 50 combined with abuse deterrent composition 40 of the present invention. ADC 40 can contain one or more gel forming agents and/or mucous membrane irritants and/or respiratory (e.g., oral or nasal) passageway tissue irritants, and/or emetics according to the teachings set forth herein. In a second step (step 2), the combination of drug 50 and ADC 40 can then be blended with other ingredients 60, e.g. disintegrants and lubricants, to form a mix 100. Lastly, in a third step (step 3) combination 100 can then be processed using conventional practices 110, e.g., compression, into a suitable unit dosage form 120, e.g. tablets.

Suitable formulations and dosage forms of the present invention include but are not limited to powders, caplets, pills, suppositories, gels, soft gelatin capsules, capsules and compressed tablets manufactured from a pharmaceutical composition of the present invention. The dosage forms can be any shape, including regular or irregular shape depending upon the needs of the artisan.

Compressed tablets including the pharmaceutical compositions of the present invention can be direct compression tablets or non-direct compression tablets. In one embodiment, a dosage form of the present invention can be made by wet granulation, and dry granulation (e.g., slugging or roller compaction). The method of preparation and type of excipients are selected to give the tablet formulation desired physical characteristics that allow for the rapid compression of the tablets. After compression, the tablets must have a number of additional attributes such as appearance, hardness, disintegrating ability, and an acceptable dissolution profile.

Choice of fillers and other excipients typically depend on the chemical and physical properties of the drug, behavior of the mixture during processing, and the properties of the final tablets. Adjustment of such parameters is understood to be within the general understanding of one skilled in the relevant art. Suitable fillers and excipients are described in more detail above.

The manufacture of a dosage form of the present invention can involve direct compression and wet and dry granulation methods, including slugging and roller compaction. However, in the present invention, it is preferred to use direct compression techniques because of the lower processing time and cost advantages.

Accordingly, and as described further below, a directly compressible pharmaceutical composition of the present invention can be designed following the teachings set forth herein that can deter one or more of a) parenteral abuse of a drug, b) inhalation abuse of a drug, and c) oral abuse of a drug.

Such compositions and dosage forms are formed according to the present invention are described. Steps for making the compositions or dosage forms include the step of providing one or more drugs and/or analgesics described above and an amount of a gel forming polymer having a desired molecular weight or viscosity as described above, and/or providing a nasal tissue irritant, and/or providing an emetic in the amounts as described above.

By controlling the molecular weight and/or viscosity of the gel forming polymer, and/or by controlling the amount of mucous membrane irritant and/or respiratory (e.g., nasal or oral) tissue irritant such that nasal tissue irritation occurs if the composition is inhaled (e.g. through the mouth or nose), and/or by controlling the amount of emetic such that emesis ensues if more than a prescribed amount of the analgesic is consumed, a therapeutic composition suitable for use to deter drug abuse can be formed. The compositions according to the present invention can deter abuse of the analgesic by (1) forming a viscous substance upon contact with a solvent such that the substance and analgesic cannot be easily drawn into a syringe and/or (2) by inducing mucous membrane irritation and/or respiratory (e.g., nasal or oral) tissue irritation if the composition is inhaled, and/or (3) by inducing emesis if more than a prescribed amount of the analgesic is consumed.

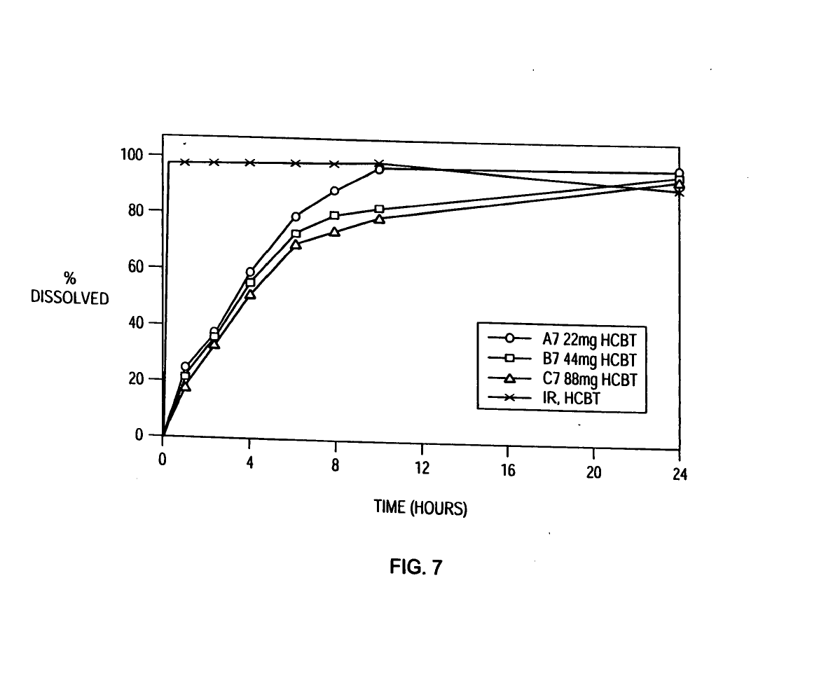

The present invention can be used to manufacture immediate release, and controlled drug release formulations. Controlled release formulations can include delayed release, bi-modal and tri-modal release, extended and sustained release oral solid dosage preparations. Examples 25 (formulation A7 of FIG. 7), 26 (formulation B7 of FIG. 7) and 27 (formulation C7 of FIG. 7) provide embodiments of the invention that can provide controlled release of a drug. The release profiles of the controlled release dosage forms of the present invention are shown in FIG. 7. The dosage forms in FIG. 7 include hydrocodone bitartrate (HCBT) as an active. As shown in FIG. 7, about 80 to 95% of the drug in a controlled release dosage form of the present invention is released after about 10 hours, as compared to an immediate release dosage form (a conventional dosage form) which is at least 75% dissolved after about 45 minutes. Other opioid formulations having an extended effect, which can be modified to further include one or more of the abuse deterrent compositions of the present invention, are described in U.S. Pat. No. 6,572,885, the entire content of which is hereby incorporated by reference. Additional embodiments of controlled release formulations for use with the present invention include the embodiments described in U.S. Patent Application Publication No. 20050020613 to Boehm et al., entitled “Sustained Release Opioid Formulations and Method of Use,” and U.S. Patent Application Publication No. 20050106249 to Hwang et al., entitled “Once-A-Day Oral Controlled Release, Oxycodone Dosage Forms,” the contents of which are hereby incorporated by reference.

Certain controlled release embodiments of the present invention can be made by first plasticizing Eudragit® and Triacetin® (glyceryl triacetate). Next oxycodone HCl, niacin, SLS, MCC and povidone can be combined in a fluid bed granulator with the plasticized Eudragit® and Triacetin®. The granulation can then be passed through a rotating impeller mill and optionally dried if the moisture content is too high. The granulation can then be waxed by melting stearyl alcohol and combining the melting stearyl alcohol with the granulation and then cooling the mixture in a fluid bed dryer. The waxed granulation can then be milled through a rotating impeller mill and blended with additional MCC, PEO, crospovidone, talc and magnesium stearate. The resulting composition can then be compressed into a dosage form, as shown in Example 44.

In another embodiment of the invention, a controlled release dosage form can be made by passing stearyl alcohol flakes through an impact mill. In step A of this embodiment, hydromorphone HCl, niacin, SLS, Eudragit®, ethylcellulose and milled stearyl alcohol are blended in a twin shell blender, and then extruded into a twin screw extruder, and resultant strands are collected on a conveyor. The strands can then be cooled on the conveyor. The cooled strands can then be cut into pellets using a pelletizer and subsequently screened. In step B of this embodiment, MCC, PEO and crospovidone are mixed in a twin shell blender. The compositions resulting from steps A and B are then combined in a twin shell blender and encapsulated, as shown in Example 45.

Another embodiment of the invention which includes subunits can be made by dispersing oxycodone HCl, niacin and PEO in a hydroalcoholic solution of hypromellose by a mechanical stirrer and applying the solution onto non-pareil seeds by a rotor granulation process to produce oxycodone HCl cores. Next, a polymer solution of ethylcellulose, polyethylene glycol, Eudragit and diethyl phthalate in ethanol can be made. Next, talc can be uniformly dispersed into the polymer solution, which is then immediately sprayed onto the oxycodone HCl cores using a Wurster process, therein completing a first subunit of the oral dosage form. A second subunit can be made by dispersing oxycodone HCl, niacin and PEO in a hydroalcoholic solution of hypromellose by mechanical stirrer, and applied onto non-pareil seeds by a rotor granulation process. Additionally, a preparation of a polymer solution of Eudragit RS, Eudragit RL, triethyl citrate and sodium lauryl sulfate in ethanol and intermixed talc can be made and immediately sprayed onto oxycodone HCl cores using a Wurster process, therein completing the second subunit of the oral dosage form. The first and second subunits can be combined in a dosage form, as described in Example 46.

Certain aspects of the present invention may be better understood as illustrated by the following examples, which are meant by way of illustration and not limitation.

### Example 1

A direct compression formulation, as shown in Table 1, for an immediate release opioid analgesic, e.g. hydrocodone bitartrate, tablet having 5 mg of hydrocodone bitartrate was formed by weighing each component separately and mixing the hydrocodone bitartrate and the polymer in a V-blender for about 5 to 10 minutes at low shear conditions or in a high shear blender by mixing 2 to 5 minutes. The other formulation excipients were added to the above blend excepting the lubricant and mixed at the same rate for additional 5 to about 10 minutes. Finally, the lubricant, magnesium stearate was added to the formulation and blended at the same rate for an additional 3 to 5 minutes. This polymeric matrix containing the drug and other excipients was further compressed on a rotary tablet press to form pharmaceutically acceptable tablets.

The tablets were monitored for weight, hardness, thickness and friability. The tablets were tested for assay, release characteristics (in-vitro dissolution method) and abuse deterrent properties.

Samples of the tablets were subjected to dissolution testing using USP Apparatus 2 (U.S. Pharmacopoeia, XXVI, 2003), speed 50 rpm at 37° C., in purified water as dissolution medium for a period of 90 minutes. The acceptable dissolution criterion is not less than 75 percent of the drug dissolved in 45 minutes.

To evaluate abuse deterrent properties of the formulation a method has been developed that mimics the street abuser's method for abuse.
(i) The tablets are crushed and the resulting powder is placed into table/teaspoon.
(ii) Measured amount of water is added to the spoon. Contents of the spoon are heated for about 1 to 2 minutes.
(iii) Contents of the spoon are withdrawn using a syringe equipped with a needle.
(iv) The volume of the sample removed from the spoon is measured and the contents of the syringe are tested for the active, using a suitable analytical test method such as UV/VIS spectrophotometry.

TABLE 1

| Component | Weight (mg)/tablet |
|---|---|
| Hydrocodone bitartrate | 5 |
| Polyvinyl alcohol | 160 |
| Avicel PH 102 | 333 |
| Starch 21 | 54 |
| Zinc sulfate | 30 |
| Explotab | 15 |
| Cab-O-Sil | 1.5 |
| Magnesium stearate | 1.5 |
| Total | 600 |

An in-vitro dissolution criterion of NLT 75% of the drug dissolved in 45 minutes was met.

The drug extracted by the abuse-test method detailed above was about 34 percent.

### Example 2

TABLE 2

| Component | Weight (mg)/tablet |
|---|---|
| Hydrocodone bitartrate | 5 |
| Polyvinyl alcohol | 160 |
| Crospovidone | 90 |
| Avicel PH 102 | 120 |
| Starch 21 | 43 |
| Zinc sulfate | 30 |
| Cab-O-Sil | 1 |
| Magnesium stearate | 1 |
| Total | 450 |

As shown by Table 2, a direct compression formulation of hydrocodone bitartrate immediate release formulation including a dosage of 5 mg of hydrocodone bitartrate was prepared and tested using the blending conditions and procedure as stated in Example 1.

An in-vitro dissolution criterion of NLT 75% of the drug dissolved in 45 minutes was met.

The drug extracted by the abuse-test method was about 31 percent.

### Example 3

TABLE 3

| Component | Weight (mg)/tablet |
|---|---|
| Hydrocodone bitartrate | 5 |
| Polyox | 70 |
| Crospovidone | 152 |
| Avicel PH 102 | 304 |
| Zinc sulfate | 150 |
| Sodium lauryl sulfate | 1 |
| Cab-O-Sil | 14 |
| Magnesium stearate | 4 |
| Total | 700 |

As shown by Table 3, a direct compression formulation of hydrocodone bitartrate immediate release formulation including a dosage of 5 mg of hydrocodone bitartrate was prepared and tested using the blending conditions and procedure as stated in Example 1.

An in-vitro dissolution criterion of NLT 75% of the drug dissolved in 45 minutes was met.

The drug extracted by the abuse-test method was about 11 percent.

### Example 4

TABLE 4

| Component | Weight (mg)/tablet |
|---|---|
| Hydrocodone bitartrate | 5 |
| Polyvinyl alcohol | 80 |
| Polyox | 15 |
| Avicel PH 102 | 300 |
| Zinc sulfate | 50 |
| Sodium lauryl sulfate | 7 |
| Crospovidone | 100 |
| Cab-O-Sil | 2 |
| Magnesium stearate | 1 |
| Total | 560 |

As shown by Table 4, a direct compression formulation of hydrocodone bitartrate immediate release formulation including a dosage of 5 mg of hydrocodone bitartrate was prepared and tested using the blending conditions and procedure as stated in Example 1.

An in-vitro dissolution criterion of NLT 75% of the drug dissolved in 45 minutes was met.

The drug extracted by the abuse-test method was about 6.5 percent.

### Example 5

TABLE 5

| Component | Weight (mg)/tablet |
|---|---|
| Hydrocodone bitartrate | 5 |
| Methocel K100 LV | 25 |
| Avicel PH 102 | 300 |
| Zinc sulfate | 50 |
| Sodium lauryl sulfate | 7 |
| Crospovidone | 100 |
| Cab-O-Sil | 2 |
| Magnesium stearate | 1 |
| Total | 490 |

As shown by Table 5, a direct compression formulation of hydrocodone bitartrate immediate release formulation including a dosage of 5 mg of hydrocodone bitartrate was prepared and tested using the blending conditions and procedure as stated in Example 1.

An in-vitro dissolution criterion of NLT 75% of the drug dissolved in 45 minutes was met.

The drug extracted by the abuse-test method was about 17 percent.

### Example 6

TABLE 6

| Component | Weight (mg)/tablet |
|---|---|
| Oxycodone hydrochloride | 5 |
| Polyox | 25 |
| Avicel PH 102 | 300 |
| Zinc sulfate | 50 |
| Sodium lauryl sulfate | 7 |
| Crospovidone | 100 |
| Cab-O-Sil | 2 |
| Magnesium stearate | 1 |
| Total | 490 |

As shown by Table 6, a direct compression formulation of oxycodone hydrochloride immediate release formulation including a dosage of 5 mg of oxycodone hydrochloride was prepared and tested using the blending conditions and procedure as stated in Example 1.

An in-vitro dissolution criterion of NLT 70% of the drug dissolved in 45 minutes was met.

The drug extracted by the abuse-test method was about 9 percent.

### Example 7

TABLE 7

| Component | Weight (mg)/tablet |
|---|---|
| Morphine sulfate | 20 |
| Polyox | 20 |
| Avicel PH 102 | 300 |
| Zinc sulfate | 50 |
| Sodium lauryl sulfate | 7 |
| Crospovidone | 100 |
| Cab-O-Sil | 2 |
| Magnesium stearate | 1 |
| Total | 500 |

As shown by Table 7, a direct compression formulation of morphine sulfate immediate release formulation including a dosage of 20 mg of morphine sulfate was prepared and tested using the blending conditions and procedure as stated in Example 1.

An in-vitro dissolution criterion of NLT 75% of the drug dissolved in 45 minutes was met.

The drug extracted by the abuse-test method was about 16 percent.

### Example 8

TABLE 8

| Component | Weight (mg)/tablet |
|---|---|
| Morphine sulfate | 20 |
| Polyvinyl alcohol | 160 |
| Avicel PH 102 | 318 |
| Zinc sulfate | 30 |
| Explotab | 30 |
| Starch 21 | 54 |
| Cab-O-Sil | 1.5 |
| Magnesium stearate | 1.5 |
| Total | 615 |

As shown by Table 8, a direct compression formulation of morphine sulfate immediate release formulation including a dosage of 20 mg of morphine sulfate was prepared and tested using the blending conditions and procedure as stated in Example 1.

An in-vitro dissolution criterion of NLT 75% of the drug dissolved in 45 minutes was met.

The drug extracted by the abuse-test method was about 12 percent.

### Example 9

TABLE 9

| Component | Weight (mg)/tablet |
|---|---|
| Morphine sulfate | 40 |
| Polyox | 15 |
| Avicel PH 102 | 300 |
| Zinc sulfate | 50 |
| Sodium lauryl sulfate | 7 |
| Crospovidone | 100 |
| Cab-O-Sil | 2 |
| Magnesium stearate | 1 |
| Total | 515 |

As shown by Table 9, a direct compression formulation of morphine sulfate immediate release formulation including a dosage of 40 mg of morphine sulfate was prepared and tested using the blending conditions and procedure as stated in Example 1.

An in-vitro dissolution criterion of NLT 75% of the drug dissolved in 45 minutes was met.

The drug extracted by the abuse-test method was about 15 percent.

### Example 10

TABLE 10

| Component | Weight (mg)/tablet |
|---|---|
| Morphine sulfate | 40 |
| Polyvinyl alcohol | 200 |
| Avicel PH 102 | 278 |
| Zinc sulfate | 30 |
| Explotab | 30 |
| Starch 21 | 54 |
| Cab-O-Sil | 1.5 |
| Magnesium stearate | 1.5 |
| Total | 635 |

As shown by Table 10, a direct compression formulation of morphine sulfate immediate release formulation including a dosage of 40 mg of morphine sulfate was prepared and tested using the blending conditions and procedure as stated in Example 1.

An in-vitro dissolution criterion of NLT 75% of the drug dissolved in 45 minutes was met.

The drug extracted by the abuse-test method was about 6 percent.

### Example 11

TABLE 11

| Component | Weight (mg)/tablet |
|---|---|
| Hydrocodone bitartrate | 7.5 |
| Polyox | 25 |
| Avicel PH 102 | 297.5 |
| Crospovidone | 100 |
| Zinc sulfate | 50 |
| Sodium lauryl sulfate | 7 |
| Cab-O-Sil | 2 |
| Magnesium stearate | 1 |
| Total | 490 |

As shown by Table 11, a direct compression formulation of hydrocodone bitartrate immediate release formulation including a dosage of 7.5 mg of hydrocodone bitartrate was prepared and tested using the blending conditions and procedure as stated in Example 1.

An in-vitro dissolution criterion of NLT 75% of the drug dissolved in 45 minutes was met.

The drug extracted by the abuse-test method was about 5 percent.

### Example 12

TABLE 12

| Component | Weight (mg)/tablet |
|---|---|
| Hydrocodone bitartrate | 10 |
| Polyvinyl alcohol | 80 |
| Polyox | 15 |
| Avicel PH 102 | 295 |
| Crospovidone | 100 |
| Zinc sulfate | 50 |
| Sodium lauryl sulfate | 7 |
| Cab-O-Sil | 2 |
| Magnesium stearate | 1 |
| Total | 560 |

As shown by Table 12, a direct compression formulation of hydrocodone bitartrate immediate release formulation including a dosage of 10 mg of hydrocodone bitartrate was prepared and tested using the blending conditions and procedure as stated in Example 1.

An in-vitro dissolution criterion of NLT 75% of the drug dissolved in 45 minutes was met.

The drug extracted by the abuse-test method was about 9.5 percent.

### Example 13

TABLE 13

| Component | Weight (mg)/tablet |
|---|---|
| Hydrocodone bitartrate | 5 |
| Carbopol 971P | 10 |
| Avicel PH 102 | 300 |
| Crospovidone | 100 |
| Zinc sulfate | 50 |
| Sodium lauryl sulfate | 7 |
| Cab-O-Sil | 2 |
| Magnesium stearate | 1 |
| Total | 490 |

As shown by Table 13, a direct compression formulation of hydrocodone bitartrate immediate release formulation including a dosage of 5 mg of hydrocodone bitartrate was prepared and tested using the blending conditions and procedure as stated in Example 1.

An in-vitro dissolution criterion of NLT 75% of the drug dissolved in 45 minutes was met.

The drug extracted by the abuse-test method was about 27 percent.

### Example 14

TABLE 14
Formulation A3

| Component | Weight (mg/tablet) |
|---|---|
| Hydrocodone Bitartrate | 5 |
| Polyvinyl Alcohol | 160 |
| Avicel PH 102 | 318 |
| Zinc Sulfate | 30 |
| Starch 21 | 54 |
| Explotab | 30 |
| Cab-O-Sil | 1.5 |
| Magnesium Stearate | 1.5 |
| Total | 600 |

As shown by Table 14, a direct compression formulation of hydrocodone bitartrate immediate release formulation including a dosage of 5 mg of hydrocodone bitartrate was prepared and tested using the blending conditions and procedure as stated in Example 1.

An in-vitro dissolution showed about 62% of the drug dissolved in 45 minutes.

The drug extracted by the abuse-test method was about 26.77 percent.

### Example 15

TABLE 15
Formulation B3

| Component | Weight (mg/tablet) |
|---|---|
| Hydrocodone Bitartrate | 5 |
| Polyvinyl Alcohol | 160 |
| Avicel PH 102 | 333 |
| Zinc Sulfate | 30 |
| Explotab | 15 |
| Starch 21 | 54 |
| Cab-O-Sil | 1.5 |
| Magnesium Stearate | 1.5 |
| Total | 600 |

As shown by Table 15, a direct compression formulation of hydrocodone bitartrate immediate release formulation including a dosage of 5 mg of hydrocodone bitartrate was prepared and tested using the blending conditions and procedure as stated in Example 1.

An in-vitro dissolution showed about 72% of the drug dissolved in 45 minutes.

The drug extracted by the abuse-test method was about 31.8 percent.

### Example 16

TABLE 16
Formulation C3

| Component | Weight (mg/tablet) |
|---|---|
| Hydrocodone Bitartrate | 5 |
| Polyvinyl Alcohol | 160 |
| Avicel PH 102 | 120 |
| Zinc Sulfate | 30 |
| Crospovidone (PVP XL) | 40 |
| Starch 21 | 43 |
| Cab-O-Sil | 1 |
| Magnesium Stearate | 1 |
| Total | 400 |

As shown by Table 16, a direct compression formulation of hydrocodone bitartrate immediate release formulation including a dosage of 5 mg of hydrocodone bitartrate was prepared and tested using the blending conditions and procedure as stated in Example 1.

An in-vitro dissolution showed about 75% of the drug dissolved in 45 minutes.

The drug extracted by the abuse-test method was about 35.75 percent.

### Example 17

TABLE 17
Formulation D3

| Component | Weight (mg/tablet) |
|---|---|
| Hydrocodone Bitartrate | 5 |
| Polyvinyl Alcohol | 160 |
| Avicel PH 102 | 120 |
| Zinc Sulfate | 30 |
| Crospovidone (PVP XL) | 100 |
| Starch 21 | 33 |
| Cab-O-Sil | 1 |
| Magnesium Stearate | 1 |
| Total | 450 |

As shown by Table 17, a direct compression formulation of hydrocodone bitartrate immediate release formulation including a dosage of 5 mg of hydrocodone bitartrate was prepared and tested using the blending conditions and procedure as stated in Example 1.

An in-vitro dissolution showed about 82% of the drug dissolved in 45 minutes.

The drug extracted by the abuse-test method was about 35.8 percent.

### Example 18

TABLE 18
Formulation E3

| Component | Weight (mg/tablet) |
|---|---|
| Hydrocodone Bitartrate | 5 |
| Polyvinyl Alcohol | 160 |
| Avicel PH 102 | 333 |
| Zinc Sulfate | 30 |
| Starch 21 | 54 |
| Crospovidone (PVP XL) | 15 |
| Cab-O-Sil | 1.5 |
| Magnesium Stearate | 1.5 |
| Total | 600 |

As shown by Table 18, a direct compression formulation of hydrocodone bitartrate immediate release formulation including a dosage of 5 mg of hydrocodone bitartrate was prepared and tested using the blending conditions and procedure as stated in Example 1.

An in-vitro dissolution showed about 79% of the drug dissolved in 45 minutes.

The drug extracted by the abuse-test method was about 42.5 percent.

### Example 19

TABLE 19
Formulation F3

| Component | Weight (mg/tablet) |
|---|---|
| Hydrocodone Bitartrate | 5 |
| Polyvinyl Alcohol | 160 |
| Avicel PH 102 | 119 |
| Zinc Sulfate | 30 |
| Crospovidone (PVP XL) | 100 |
| Starch 21 | 33 |
| Cab-O-Sil | 1 |
| Magnesium Stearate | 2 |
| Total | 450 |

As shown by Table 19, a direct compression formulation of hydrocodone bitartrate immediate release formulation including a dosage of 5 mg of hydrocodone bitartrate was prepared and tested using the blending conditions and procedure as stated in Example 1.

An in-vitro dissolution criterion of NLT 75% of the drug dissolved in 45 minutes was met.

The drug extracted by the abuse-test method was about 54 percent.

### Example 20

TABLE 20

| Component | Weight (mg/tablet) |
|---|---|
| Hydrocodone | 5 |
| Bitartrate | |
| Polyvinyl Alcohol | 95 |
| Avicel PH 102 | 192 |
| Zinc Sulfate | 30 |
| Starch 21 | 140 |
| Ac-Di-Sol | 35 |
| Cab-O-Sil | 1 |
| Magnesium Stearate | 2 |
| Total | 500 |

As shown in Table 20, a direct compression formulation of hydrocodone bitartrate immediate release formulation including a dosage of 5 mg of hydrocodone bitartrate was prepared and tested using the blending conditions and procedure as stated in Example 1.

An in-vitro dissolution criterion of NLT 75% of the drug dissolved in 45 minutes was met.

The drug extracted by the abuse-test method was about 60 percent.

### Example 21

TABLE 21

| Component | Weight (mg/tablet) |
|---|---|
| Oxycodone Hydrochloride | 5 |
| Avicel PH 102 | 119 |
| Zinc Sulfate | 30 |
| Crospovidone (PVP XL) | 100 |
| Starch 21 | 33 |
| Cab-O-Sil | 1 |
| Magnesium Stearate | 2 |
| Total | 290 |

As shown by Table 21, a direct compression formulation of hydrocodone bitartrate immediate release formulation including a dosage of 5 mg of hydrocodone bitartrate was prepared and tested using the blending conditions and procedure as stated in Example 1.

An in-vitro dissolution criterion of NLT 75% of the drug dissolved in 45 minutes was met.

The drug extracted by the abuse-test method was about 94 percent.

### Example 22

TABLE 22

| | Weight |
|---|---|
| Component | (mg/tablet) |
| Hydrocodone | 5 |
| Bitartrate | |
| Polyvinyl Alcohol | 50 |
| Avicel PH 102 | 192 |
| Zinc Sulfate | 30 |
| Starch 21 | 140 |
| Ac-Di-Sol | 35 |
| Cab-O-Sil | 1 |
| Magnesium Stearate | 2 |
| Total | 455 |

As shown in Table 22, a direct compression formulation of hydrocodone bitartrate immediate release formulation including a dosage of 5 mg of hydrocodone bitartrate was prepared and tested using the blending conditions and procedure as stated in Example 1.

An in-vitro dissolution criterion of NLT 75% of the drug dissolved in 45 minutes was met.

The drug extracted by the abuse-test method was about 70 percent.

### Example 23

TABLE 23

| Component | Weight (mg/tablet) |
|---|---|
| Hydrocodone | 5 |
| Bitartrate | |
| Polyvinyl Alcohol | 160 |
| Avicel PH 102 | 318 |
| Zinc Sulfate | 30 |
| Explotab | 30 |
| Cab-O-Sil | 1.5 |
| Magnesium Stearate | 1.5 |
| Total | 600 |

As shown in Table 23, a direct compression formulation of hydrocodone bitartrate immediate release formulation including a dosage of 5 mg of hydrocodone bitartrate was prepared and tested using the blending conditions and procedure as stated in Example 1.

An in-vitro dissolution criterion of NLT 75% of the drug dissolved in 45 minutes was met.

The drug extracted by the abuse-test method was about 33 percent.

### Example 24

TABLE 24

| Component | Weight (mg/tablet) |
|---|---|
| Hydrocodone | 10 |
| Bitartrate | |
| Avicel PH 102 | 318 |
| Zinc Sulfate | 50 |
| Crospovidone (PVP XL) | 100 |
| Sodium Lauryl Sulfate | 7 |
| Cab-O-Sil | 1.5 |
| Magnesium Stearate | 1.5 |
| Total | 488 |

As shown in Table 24, a direct compression formulation of hydrocodone bitartrate immediate release formulation including a dosage of 5 mg of hydrocodone bitartrate was prepared and tested using the blending conditions and procedure as stated in Example 1.

An in-vitro dissolution criterion of NLT 75% of the drug dissolved in 45 minutes was met.

The drug extracted by the abuse-test method was about 85 percent.

### Example 25

TABLE 25
Formulation A7

| Component | Weight (mg/tablet) |
|---|---|
| Hydrocodone Bitartrate | 22 |
| Polyvinyl Alcohol | 250 |
| Cab-O-Sil | 1.38 |
| Magnesium Stearate | 2.76 |
| Total | 276.14 |

An in-vitro dissolution showed about 98% dissolution after 10 hours.

### Example 26

TABLE 26
Formulation B7

| Component | Weight (mg/tablet) |
|---|---|
| Hydrocodone Bitartrate | 44 |
| Polyvinyl Alcohol | 450 |
| Cab-O-Sil | 1.5 |
| Magnesium Stearate | 2.0 |
| Total | 497.5 |

An in-vitro dissolution showed about 82% dissolution after 10 hours.

### Example 27

TABLE 27
Formulation C7

| Component | Weight (mg/tablet) |
|---|---|
| Hydrocodone Bitartrate | 88 |
| Polyvinyl Alcohol | 600 |
| Cab-O-Sil | 1.5 |
| Magnesium Stearate | 2.0 |
| Total | 691.5 |

An in-vitro dissolution showed about 80% dissolution after 10 hours.

### Example 28

TABLE 28

| Component | Weight (mg)/tablet |
|---|---|
| Oxycodone | 5 |
| hydrochloride | |
| Polyox | 25 |
| Avicel PH 102 | 250 |
| Zinc sulfate | 100 |
| Sodium lauryl sulfate | 7 |
| Crospovidone | 100 |
| Cab-O-Sil | 2 |
| Magnesium stearate | 1 |
| Total | 490 |

As shown by Table 28, an immediate release direct compression formulation containing 5 mg of oxycodone hydrochloride was prepared using the blending conditions and procedure as stated in Example 1.

### Example 29

TABLE 29

| Component | Weight (mg)/tablet |
|---|---|
| Oxycodone hydrochloride | 5 |
| Polyox | 25 |
| Avicel PH 102 | 200 |
| Zinc sulfate | 150 |
| Sodium lauryl sulfate | 7 |
| Crospovidone | 100 |
| Cab-O-Sil | 2 |
| Magnesium stearate | 1 |
| Total | 490 |

As shown by Table 29, an immediate release direct compression formulation containing 5 mg of oxycodone hydrochloride was prepared using the blending conditions and procedure as stated in Example 1.

### Example 30

TABLE 30

| Component | Weight (mg)/tablet |
|---|---|
| Oxycodone hydrochloride | 5 |
| Polyox | 25 |
| Avicel PH 102 | 300 |
| Niacin | 50 |
| Sodium lauryl sulfate | 7 |
| Crospovidone | 100 |
| Cab-O-Sil | 2 |
| Magnesium stearate | 1 |
| Total | 490 |

In light of the teachings set forth herein, an embodiment of the invention having the above described composition can be made.

### Example 31

TABLE 31

| Component | Weight (mg)/tablet |
|---|---|
| Oxycodone hydrochloride | 5 |
| Polyox | 25 |
| Avicel PH 102 | 400 |
| Niacin | 100 |
| Sodium lauryl sulfate | 7 |
| Crospovidone | 100 |
| Cab-O-Sil | 2 |
| Magnesium stearate | 1 |
| Total | 640 |

In light of the teachings set forth herein, an embodiment of the invention having the above described composition can be made.

### Example 32

TABLE 32

| Component | Weight (mg)/tablet |
|---|---|
| Oxycodone hydrochloride | 5 |
| Polyox | 25 |
| Avicel PH 102 | 300 |
| Docusate Sodium | 85 |
| Niacin | 100 |
| Crospovidone | 100 |
| Cab-O-Sil | 2 |
| Magnesium stearate | 1 |
| Total | 625 |

In light of the teachings set forth herein, an embodiment of the invention having the above described composition can be made.

### Example 33

TABLE 33

| Component | Weight (mg)/tablet |
|---|---|
| Oxycodone hydrochloride | 5 |
| Polyox | 25 |
| Avicel PH 102 | 300 |
| Turmeric | 25 |
| Sodium lauryl sulfate | 7 |
| Crospovidone | 100 |
| Cab-O-Sil | 2 |
| Magnesium stearate | 1 |
| Total | 465 |

In light of the teachings set forth herein, an embodiment of the invention having the above described composition can be made.

### Example 34

TABLE 34

| Component | Weight (mg)/tablet |
|---|---|
| Oxycodone hydrochloride | 5 |
| Polyox | 25 |
| Avicel PH 102 | 300 |
| Niacin | 100 |
| FD & C Green #3 | 5 |
| Sodium lauryl sulfate | 7 |
| Crospovidone | 100 |
| Cab-O-Sil | 2 |
| Magnesium stearate | 1 |
| Total | 545 |

In light of the teachings set forth herein, an embodiment of the invention having the above described composition can be made.

### Example 35

TABLE 35

| Component | Weight (mg)/tablet |
|---|---|
| Polyox | 25 |
| Avicel PH 102 | 340 |
| Niacin | 15 |
| Sodium lauryl sulfate | 7 |
| Crospovidone | 100 |
| Cab-O-Sil | 2 |
| Magnesium stearate | 1 |
| Total | 490 |

In light of the teachings set forth herein, an embodiment of the invention (excluding a drug susceptible to abuse) having the above described composition can be made.

### Example 36

TABLE 36

| Component | Weight (mg)/tablet |
|---|---|
| Polyox | 25 |
| Avicel PH 102 | 325 |
| Niacin | 30 |
| Sodium lauryl sulfate | 7 |
| Crospovidone | 100 |
| Cab-O-Sil | 2 |
| Magnesium stearate | 1 |
| Total | 490 |

In light of the teachings set forth herein, an embodiment of the invention (excluding a drug susceptible to abuse) having the above described composition can be made.

### Example 37

TABLE 37

| Component | Weight (mg)/tablet |
|---|---|
| Polyox | 25 |
| Avicel PH 102 | 310 |
| Niacin | 45 |
| Sodium lauryl sulfate | 7 |
| Crospovidone | 100 |
| Cab-O-Sil | 2 |
| Magnesium stearate | 1 |
| Total | 490 |

In light of the teachings set forth herein, an embodiment of the invention (excluding a drug susceptible to abuse) having the above described composition can be made.

### Example 38

TABLE 38

| Component | Weight (mg)/tablet |
|---|---|
| Polyox | 25 |
| Avicel PH 102 | 295 |
| Niacin | 60 |
| Sodium lauryl sulfate | 7 |
| Crospovidone | 100 |
| Cab-O-Sil | 2 |
| Magnesium stearate | 1 |
| Total | 490 |

In light of the teachings set forth herein, an embodiment of the invention (excluding a drug susceptible to abuse) having the above described composition can be made.

### Example 39

TABLE 39

| Component | Weight (mg)/tablet |
|---|---|
| Polyox | 25 |
| Avicel PH 102 | 280 |
| Niacin | 75 |
| Sodium lauryl sulfate | 7 |
| Crospovidone | 100 |
| Cab-O-Sil | 2 |
| Magnesium stearate | 1 |
| Total | 490 |

In light of the teachings set forth herein, an embodiment of the invention (excluding a drug susceptible to abuse) having the above described composition can be made.

### Example 40

TABLE 40

| Component | Weight (mg)/tablet |
|---|---|
| Polyox | 25 |
| Avicel PH 102 | 355 |
| Sodium lauryl sulfate | 7 |
| Crospovidone | 100 |
| Cab-O-Sil | 2 |
| Magnesium stearate | 1 |
| Total | 490 |

In light of the teachings set forth herein, an embodiment of the invention (excluding a drug susceptible to abuse) having the above described composition can be made.

### Example 41

TABLE 41

| Component | Weight (mg)/tablet |
|---|---|
| Polyox | 25 |
| Avicel PH 102 | 332 |
| Niacin | 30 |
| Crospovidone | 100 |
| Cab-O-Sil | 2 |
| Magnesium stearate | 1 |
| Total | 490 |

In light of the teachings set forth herein, an embodiment of the invention (excluding a drug susceptible to abuse) having the above described composition can be made.

### Example 42

TABLE 42

| Component | Weight (mg)/tablet |
|---|---|
| Polyox | 25 |
| Avicel PH 102 | 317 |
| Niacin | 45 |
| Crospovidone | 100 |
| Cab-O-Sil | 2 |
| Magnesium stearate | 1 |
| Total | 490 |

In light of the teachings set forth herein, an embodiment of the invention (excluding a drug susceptible to abuse) having the above described composition can be made.

### Example 43

TABLE 43

| Component | Weight (mg)/tablet |
|---|---|
| Oxycodone Hydrochloride | 5 |
| Polyox | 25 |
| Avicel PH 102 | 327 |
| Niacin | 30 |
| Crospovidone | 100 |
| Cab-O-Sil | 2 |
| Magnesium stearate | 1 |
| Total | 490 |

In light of the teachings set forth herein, an embodiment of the invention having the above described composition can be made.

### Example 44

TABLE 44

| Component | Weight (mg)/tablet |
|---|---|
| Oxycodone | 5 |
| Polyox | 25 |
| Avicel PH 102 | 320 |
| Niacin | 30 |
| Sodium lauryl sulfate | 7 |
| Crospovidone | 100 |
| Cab-O-Sil | 2 |
| Magnesium stearate | 1 |
| Total | 490 |

In light of the teachings set forth herein, an embodiment of the invention (excluding a drug susceptible to abuse) having the above described composition can be made.

### Example 45

TABLE 45

| Component | Weight (mg)/tablet |
|---|---|
| Oxycodone HC1 | 20 |
| Niacin | 120 |
| Sodium Lauryl Sulfate | 7 |
| Microcystalline Cellulose (part 1) | 60 |
| Povidone | 5 |
| Eudragit RS 30D (dry wt.) | 10 |
| Triacetin | 2 |
| Stearyl Alcohol | 25 |
| Microcystalline Cellulose (part 2) | 162.2 |
| Polyethylene Oxide | 25 |
| Crospovidone | 50 |
| Talc | 2.5 |
| Magnesium Stearate | 1.3 |
| Purified Water | 34* |
| Total | 490 |

* Remains in product as residual moisture only

In light of the teachings set forth herein, an embodiment of the invention having the above described composition can be made.

### Example 46

Hydromorphone HCl Controlled Release Capsules

TABLE 46

| Component | Weight (mg)/tablet |
|---|---|
| Hydromorphone HC1 | 16 |
| Niacin | 120 |
| Sodium Lauryl Sulfate | 7 |
| Eudragit RSPO | 76 |
| Ethylcellulose | 4.5 |
| Stearyl Alcohol | 27 |
| Microcystalline Cellulose | 195 |
| Polyethylene Oxide | 35 |
| Crospovidone | 50 |
| Hard Gelatin Capsules | |
| Total | 530.5 |

In light of the teachings set forth herein, an embodiment of the invention having the above described composition can be made.

### Example 47

Oxycodone HCl Controlled Release Capsules

TABLE 47

| Component | Weight (mg)/tablet |
|---|---|
| First subunit | |
| Oxycodone HC1 | 40 |
| Niacin | 120 |
| Polyethylene Oxide | 20 |
| Non-pareil seed (#16-18 mesh) | 131.9 |
| Hypromellose | 3.3 |
| Ethylcellulose | 19.9 |
| Polyethylene glycol 6000 | 6.7 |
| Eudragit L100-55 | 5.6 |
| Diethyl phthalate | 3.9 |
| Talc | 17.6 |
| Total | 368.9 |
| Second subunit | |
| Oxycodone HC1 | 40 |
| Niacin | 120 |
| Polyethylene Oxide | 20 |
| Non-pareil seed (#20-25 mesh) | 128.9 |
| Hypromellose | 6.3 |
| Eudragit RS PO | 54.1 |
| Eudragit RL PO | 1.9 |
| Triethyl citrate | 5.4 |
| Sodium lauryl sulfate | 10.0 |
| Talc | 30.9 |
| Total | 417.5 |

In light of the teachings set forth herein, an embodiment of the invention having the above described composition can be made.

It will be appreciated by persons skilled in the art that numerous variations and/or modifications may be made to the invention shown in the specific embodiments without departing form the spirit and scope of the invention as broadly described. Further, each and every reference cited above is hereby incorporated by reference as if fully set forth herein.

What is claimed is:

1. A pharmaceutical composition comprising:
1. pseudoephedrine;
2. sodium lauryl sulfate;
3. croscarmellose sodium;
4. a gel forming polymer;
5. polyethylene glycol; and
6. an acrylic polymer.

2. The composition of claim 1, wherein the gel forming polymer comprises hydroxypropylmethylcellulose.

3. The composition of claim 1, further comprising hydroxypropyl cellulose.

4. The composition of claim 1, wherein the sodium lauryl sulfate, crosscarmellose sodium, and gel forming polymer form a first immediate release sequestered subunit.

5. The composition of claim 4, wherein the pseudoephedrine is contained in a second immediate release sequestered subunit.

6. The composition of claim 5, wherein the first immediate release sequestered subunit and the second immediate release sequestered subunit are homogenously mixed.

7. The composition of claim 1, wherein the acrylic polymer comprises a cationic copolymer based on dimethylaminoethyl methacrylate, butyl methacrylate, and methyl methacrylate.

8. The composition of claim 1, further comprising microcrystalline cellulose.

9. The composition of claim 1, wherein the composition is a chewable pharmaceutical product.

10. The composition of claim 1, wherein the sodium lauryl sulfate is present in an amount of about 1 wt % to about 20 wt % of the composition.

11. The composition of claim 1, wherein the croscarmellose sodium is present in an amount of about 2 wt % to about 25 wt %.

12. The composition of claim 1, wherein the gel forming polymer is present in an amount of about 3 wt % to about 70 wt %.

13. The composition of claim 1, wherein the polyethylene glycol is present in an amount of about 0.1 wt % to about 2 wt %.
